(19) United States
(12) Patent Application Publication
(10) Pub. No.: US 2023/0059683 A1
(43) Pub. Date: Feb. 23, 2023

(54) TRANSPOSITION-BASED DIAGNOSTICS METHODS AND DEVICES

(71) Applicant: THE TRUSTEES OF COLUMBIA UNIVERSITY IN THE CITY OF NEW YORK, New York, NY (US)

(72) Inventors: Samuel K. Sia, New York, NY (US); Samuel Sternberg, New York, NY (US); Siddarth Arumugam, New York, NY (US); Kenneth Shepard, New York, NY (US)

(21) Appl. No.: 17/758,415

(22) PCT Filed: Jan. 7, 2021
(86) PCT No.: PCT/US2021/012484

§ 371 Date: Jul. 6, 2022

Related U.S. Application Data
(60) Provisional application No. 62/981,916, filed on Feb. 26, 2020.
(60) Provisional application No. 62/958,083, filed on Jan. 7, 2020.

(51) Int. Cl. C12N 15/10; C12N 15/113; C12N 9/22; C12Q 1/6897; G01N 33/53; G01N 33/543

(52) U.S. Cl. CPC C12N 15/102; C12N 15/113; C12N 9/22; C12Q 1/6897; G01N 33/5308; G01N 33/543; C12N 2310/20

(57) ABSTRACT

The present disclosure provides devices, systems, and methods for detection of nucleic acids based on CRISPR-Cas editing systems, for example for use in biosurveillance. Disclosed herein are systems and methods utilizing two devices: 1) a point of—need disposable “FET Strip” (enzymatic), and 2) an instrument-operated “FET Multiplexor” (electronic), to provide detection of a nucleic acid for biosurveillance.

### CROSS-REFERENCE TO RELATED APPLICATIONS

This application claims the benefit of U.S. Provisional Application No. 62/958,083, filed Jan. 7, 2020, and U.S. Provisional Application No. 62/981,916, filed Feb. 26, 2020, the entire contents of each are incorporated herein by reference.

### FIELD

The present disclosure provides devices, systems, and methods for detection of nucleic acids.

### BACKGROUND

With rapid identification of pathogens being critical to timely implementation of countermeasures, there exists an urgent and unmet need for robust field-deployable strategies for rapid detection of emergent and engineered pathogens. In the current state of field-deployable nucleic-acid diagnostic technologies for disease biosurveillance, advances have been made to reduce the complexity of high-performing microfluidic devices, but they are usually not as simple as lateral flow assay (LFA) tests, which in turn are limited in sensitivity unless combined with target pre-amplification or additional complex steps. More recently, diagnostic efforts have been boosted by gene editing, which make use of Cas enzymes that efficiently scan a genomic DNA for specific sequences. However, current methods exhibit challenges. First, to attain sensitivity, they require target pre-amplification, and, because the Cas-mediated cutting event is not directly detected, also require downstream amplification. Second, for time to result, denaturation with heating equipment and other manual steps are often needed, and the reporting method of non-specific degradation takes time. Third, only limited methods have been proposed to work on both RNA and DNA targets. Fourth, rapid reconfigurability has yet to be demonstrated.

### SUMMARY

Disclosed herein are systems and methods utilizing two devices: 1) a point-of-need disposable “FET Strip” (enzymatic), and 2) an instrument-operated “FET Multiplexor” (electronic), to provide detection of a nucleic acid for biosurveillance. The systems and methods may contain at least one or all of: 1) Cas 12, Cas 13, and/or a RNA-guided transposase that cuts and pastes donor sequences into dsDNA targets; 2) for the transposase strategy. RNA-based switches in which recognition of target RNA exposes a cryptic guide RNA (gRNA), leading to detectable transposition events; 3) for FET Strip, a colorimetric lateral flow assay (LFA) with multiple (e.g., 10) zones for target capture and detection; 4) for FET Multiplexor, an array of single-molecule field-effect transistors (smFET) that are ultrasensitive, capable of detecting single-molecule binding events even without a reporter; 5) for FET Multiplexor, hydraulic microfluidic valves that enable fluidic handling and induce rapid surface-binding kinetics, as operated by a compact battery-powered instrument; and 6) a reporter screen and deep learning-based framework for designing guide RNAs (gRNAs), as shown in FIG. 1.

In some embodiments, the systems and methods comprise a reporter nucleic acid comprising a tag; at least one or both of a guide RNA that comprises a nucleic acid sequence complementary to a gRNA target nucleic acid sequence and an RNA switch; and a CRISPR RNA-guided Cas-transposase system, or a Cas protein selected from Cas 12a, Cas 13, and combinations thereof.

In some embodiments, the reporter nucleic acid comprises dsDNA. In some embodiments, the tag is an enzyme, an affinity tag, a single-stranded oligonucleotide, a halogen, or any combination thereof conjugated to the reporter nucleic acid.

The CRISPR RNA-guided Cas-transposase system may comprise at least one Cas protein, at least one transposase protein, or combinations thereof. In some embodiments, the CRISPR RNA-guided Cas-transposase system is derived from Type 1 CRISPR-Cas system or a Type V CRISPR-Cas system. In some embodiments, the CRISPR RNA-guided Cas-transposase system comprises Cas12k. In some embodiments, the CRISPR RNA-guided Cas-transposase system comprises Cas5, Cas6, Cas7, Cas8, or any combination thereof. In some embodiments, the CRISPR RNA-guided Cas-transposase system comprises TnsA, TnsB, TnsC, or any combination thereof.

The systems and methods may further comprise a target nucleic acid. In some embodiments, the target nucleic acid is double-stranded DNA or single-stranded RNA. In some embodiments, the single-stranded RNA target nucleic acid is complementary to at least a portion of the RNA switch. In some embodiments, the double-stranded DNA target nucleic acid is configured to receive the reporter nucleic acid as a result of a transposition. In some embodiments, the double-stranded DNA target nucleic acid comprises the gRNA target nucleic acid sequence. In some embodiments, the target nucleic acid is in a biological sample. In some embodiments, the target nucleic acid is a nucleic acid from or derived from an infectious agent.

The systems and methods may comprise a recipient nucleic acid. In some embodiments, the recipient nucleic acid is double-stranded DNA. In some embodiments, the recipient nucleic acid is configured to receive the reporter nucleic acid as a result of a transposition. In some embodiments, the recipient nucleic acid comprises the gRNA target nucleic acid sequence.

The reporter nucleic acid, the guide RNA, the RNA switch, or any combination thereof may be conjugated to a surface. In some embodiments, the reporter nucleic acid, the guide RNA, the RNA switch, or any combination thereof are conjugated to a microparticle. In some embodiments, the reporter nucleic acid and the guide RNA or the RNA switch are conjugated to the same microparticle. In some embodiments, the reporter nucleic acid is conjugated to the microparticle with a linker comprising a single-stranded polynucleotide.

The systems and methods may further comprise an indicator nucleic acid, wherein at least a portion of the indicator nucleic acid is complementary to at least a portion of the reporter nucleic acid or the single-stranded oligonucleotide tag.

The systems and methods may further comprise a linear flow assay strip. In some embodiments, the linear flow assay strip comprises a membrane and a sample filter pad on top of a portion of the membrane at first end, wherein the sample filter pad is comprised of pores smaller than those of the microparticle. In some embodiments, the indicator nucleic acid is immobilized in the membrane of the linear flow assay strip.

The systems and methods may further comprise a multiplexor chip. In some embodiments, the reporter nucleic acid, the guide RNA, the RNA switch, or any combination thereof are conjugated to the multiplexor chip. In some embodiments, the reporter nucleic acid is conjugated to the multiplexor chip. The systems and methods may further comprise a field-effect transistor biosensor. In some embodiments, the field-effect transistor biosensor is a single-molecule field-effect transistor sensor.

The methods may comprise: obtaining a target nucleic acid: incubating the sample with: a reporter nucleic acid comprising a tag, at least one or both of: a guide RNA that comprises a nucleic acid sequence complementary to a target DNA sequence and an RNA switch, and a CRISPR RNA-guided Cas-transposase system, or a Cas protein selected from Cas 12a, Cas 13, and combinations thereof to form a sample mixture: and measuring the presence of the reporter nucleic acid on a linear flow assay strip or a multiplexor chip. The methods may further comprise incubating the sample with a recipient nucleic acid. The methods may further comprise incubating the sample mixture with an indicator nucleic acid.

Other aspects and embodiments of the disclosure will be apparent in light of the following detailed description.

### BRIEF DESCRIPTION OF THE DRAWINGS

FIG. 1 is an overall scheme of FET (Fluidic Enzymatic/Electronic Tag-based) Detector, using Cas 12a/Cas 13 or RNA-guided transposase.

FIGS. 2A and 2B are detailed schemes for Cas12a/Cas13 cleavage (FIG. 2A) and RNA-guided transposition (FIG. 2B). Both schemes effectively detect both dsDNA and ssRNA targets, and for both LFA (FET Strip) and smFET CMOS (FET Multiplexor) devices.

FIGS. 3A-3D show the high-throughput assays for biochemical profiling of Cas12, Cas13, and RNA-guided transposases. FIG. 3A is a schematic of high-throughput fluorescence-based readout for Cas12a and Cas13 activity, dsDNA or ssRNA targets are recognized via target-activated collateral cleavage of fluorophore-quencher substrates, which elicits an increase in fluorescence. FIG. 3B is a schematic of RNA-guided DNA integration catalyzed by CRISPR-transposons. Target DNA binding (blue) by either QCascade (Type I) or Cas12k (Type V) results in downstream integration of donor DNA (purple). FIG. 3C shows successful recombinant expression and purification of all RNA-guided transposase components for the V. cholerae CRISPR-transposon system. FIG. 3D is a schematic of high-throughput NGS-based assay for RNA-guided transposases. A library of target DNA sequences is ligated into the substrate and subjected to biochemical integration experiments. Successful integration is assessed by targeted PCR using primer pairs that selectively amplify target-donor DNA chimeras. Because the resulting next-generation sequencing (NGS) amplifies across the target DNA sequence, this approach characterizes the relative integration efficiency for all library members in the experiment.

FIGS. 4A-4C show modified gRNAs for surface immobilization. FIG. 4A is a crystal structure of AsCas12a (Yamano et al., Cell 165, 949-962 (2016); PDB ID: 5B43), highlighting the exposed 5′-OH of the mature gRNA (AAUUUCUACUCUUGUAGAU SEQ ID NO: 1) in the right panel. 5′-extensions are engineered at this position for surface immobilization. FIG. 4B is a crystal structure of LbaCas13a (Knott et al., Nat Strucr Mol Biol 24, 825-833 (2017); PDB ID: 5W1I), highlighting the exposed 5′-OH of the mature gRNA (AAGAUAGCCCAAGAAAGAGGGCAAUAAC SEQ ID NO: 2) in the right panel. 5′-extensions are engineered at this position for surface immobilization. FIG. 4C is a cryoEM structure of VchQCascade (Halpin-Healy et al., Nature 577, 271-274 (2020); PDB ID: 6PIF), highlighting the exposed 3′-phosphate (PO₄) of the mature gRNA (GUGAACUGCCGAGUAGGUAG SEQ ID NO: 3) in the right panel. 3′-extensions arc engineered at this position for surface immobilization.

FIG. 5 is schematic of predicting CRISPR gRNA targeting efficiency with a hybrid deep neural network model integrating sequence and structure information of the gRNA and the target.

FIG. 6A is an illustration of the diazonium chemistry reaction for an smFET, whereby the CNT surface donates an electron to the positively charged N₂ group to form a covalent attachment. The hexafluorophosphate (PF₆) counterion provides stability to the FBDP molecule. The free (aldehyde) end of the molecule is available for bioconjugation. FIG. 6B is current change in smFET nanotube during exposure to diazonium reaction. Inset shows stepwise decreases potentially indicative of individual defects.

FIG. 7 is electronic binding signals at varying temperature and bias. Target probe sequence from the Ebola Virus was measured in IX PBS using 100 nM concentration of complementary RNA.

### DETAILED DESCRIPTION

The disclosed devices and related systems and methods advance the sensitivity and speed in gene-editing diagnostics to improve biosurveillance and control disease outbreaks.

Cas 12a/Cas 13 enzymes, which upon recognition of dsDNA and ssRNA targets, cleave a large number of neighboring reporter sequences containing enzymes and affinity tags, which can subsequently be detected.

RNA-guided transposase, which upon recognition of target, performs either a “cut and paste” or “copy and paste” transposition for direct insertion of unique oligonucleotide tags (and enzyme, in the case of LFA device) tags into targets. In contrast to Cas nucleases, which only cleave target sequences, CRISPR RNA-guided transposases (including Cas5/Cas6/Cas7/Cas8 and Cas12k proteins) recognize a dsDNA sequence as complementary to gRNA and cut and insert a donor DNA into the dsDNA strand via a single step, with simultaneous recognition and insertion.

Gene-editing approaches using either Cas12a/Cas 13 enzymes or an RNA-guided transposase can enable downstream unique target identification (by hybridization of complementary oligonucicotide tags) and dramatically amplify signal (by inserting DNA-enzyme conjugates for LFA device). The genomic targets made of dsDNA and ssRNA can be detected without denaturation.

An exemplary FET Multiplexor contains a 1000-plex array of ultrasensitive smFETs on a CMOS measurement substrate, with each smFET modified with nucleic acids that can be traced to each unique target, leading to rapid, sensitive, and unique identification. Using microfluidic valves actuated by solenoids which press down on liquid-filled control channels, fluid motion is controlled, including inducing rapid binding kinetics, in the FET Multiplexor. All capabilities in the multiplexor, including communication, can be run by a battery-powered instrument. This fluidic capability also aids on-the-fly reconfiguration (e.g., loading of beads and smFETs with new gRNAs).

The proposed capabilities of both systems and methods, in speed and sensitivity, are exceptional compared to previous gene-editing based diagnostics, or point-of-care diagnostics in general.

Section headings as used in this section and the entire disclosure herein are merely for organizational purposes and are not intended to be limiting. 1. Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, a “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey. Biochemistry, 41(14): 4503-4510 (2002)) and U.S. Pat. No. 5,034,506), locked nucleic acid (LNA; see Wahlestedt et al., Proc. Natl. Acad. Sci. U.S.A., 97: 5633-5638 (2000)), cyclohexenyl nucleic acids (see Wang, J. Am. Chem. Soc., 122: 8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide” “nucleotide sequence,” and “oligonucleotide” are used interchangeably. They refer to a polymeric form of nucleotides of any length, either deoxyribonucleotides or ribonucleotides, or analogs thereof.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%. 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1 FSSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

2. Systems for Detection of Nucleic Acids.

The present disclosure provides systems or kits (e.g., reagents, computer software, instruments, etc.) for detection of nucleic acids. In some embodiments, the system comprises: a reporter nucleic acid; at least one or both of: a guide RNA (gRNA) and an RNA switch; and a CRISPR RNA-guided Cas-transposase system, or a Cas protein selected from Cas 12a, Cas 13, and combinations thereof.

In some embodiments, the CRISPR RNA-guided Cas-transposase system comprises at least one Cas protein, at least one transposase protein, or a combination thereof. The CRISPR RNA-guided Cas-transposase system may comprise any combination of Cas proteins and transposase protein capable of carrying out “cut and paste” or “copy and paste” transposition. See for example, U.S. patent application Ser. No. 16/812,138, incorporated herein by reference in its entirety. The CRISPR RNA-guided Cas-transposase system, may be derived from a Class 1 CRISPR-Cas system or a Class 2 CRISPR-Cas system. The present system may be derived from a Type I CRISPR-Cas system, a Type II CRISPR-Cas system, or a Type V CRISPR-Cas system. In some embodiments, the CRISPR RNA-guided Cas-transposase system comprises Cas12k. In some embodiments, the CRISPR RNA-guided Cas-transposase system comprises Cas5, Cas6, Cas7. Cas8, or any combination thereof. For example, the CRISPR RNA-guided Cas-transposase system may comprise a Cas5/Casa fusion protein. In some embodiments, the CRISPR RNA-guided Cas-transposase system comprises TnsA, TnsB, TnsC, or any combination thereof.

The gRNA comprises a nucleic acid sequence complementary to a target nucleic acid sequence. The guide RNA sequence specifies the target nucleic acid sequence with an approximate 20-nucleotide guide sequence that directs Watson-Crick base pairing to a target sequence. The gRNA may be a non-naturally occurring gRNA. The gRNA may be a crRNA, crRNA/tracrRNA (or single guide RNA, sgRNA). The tenns “gRNA,” “guide RNA” and “guide sequence” may be used interchangeably throughout and refer to a nucleic acid comprising a sequence that determines binding specificity.

The gRNA or portion thereof that hybridizes to the target nucleic acid sequence may be between 15-40 nucleotides, or longer, in length, gRNAs or sgRNA(s) can be between about 5 and 100 nucleotides long, or longer. To facilitate gRNA design, many computational tools have been developed (Sec Prykhozhij et al. (PLoS ONE, 10(3): (2015)); Zhu et al. (PLoS ONE, 9(9) (2014)); Xiao et al. (Bioinformatics. Jan 21 (2014)); Heigwer et al. (Nat Methods, 11(2): 122-123 (2014)). Methods and tools for guide RNA design are discussed by Zhu (Frontiers in Biology, 10 (4) pp 289-296 (2015)), which is incorporated by reference herein. Additionally, there are many publicly available software tools that can be used to facilitate the design of sgRNA(s); including but not limited to. Genscript Interactive CRISPR gRNA Design Tool, WU-CRISPR, and Broad Institute GPP sgRNA Designer. Furthermore, a machine-learning framework as described herein may be used to design the gRNAs.

An RNA switch is an RNA molecule that comprises a gRNA sequence which is hidden due to the conformation of the remaining portion of the RNA switch. Upon binding to a complementary RNA sequence, the conformation of the RNA switch is altered such that the gRNA sequence is exposed and able to recognize a target nucleic acid sequence. Thus, the RNA switch can adopt at least two different structures, one which enables the gRNA sequence to bind and recognize a target nucleic acid sequence and related enzyme, and the other which does not. In some embodiments, a portion of the RNA switch is complementary to a single-stranded target nucleic acid. In some embodiments, the single-stranded target nucleic acid is RNA. The portion of the RNA switch complementary to the single-stranded target nucleic acid is separate from the portion of the RNA switch comprising the gRNA sequence.

The reporter nucleic acid may be DNA or RNA and may comprise single-stranded nucleic acids, double-stranded nucleic acids, or a combination thereof. In some embodiments, the reporter nucleic acid comprises double-stranded DNA. The reporter nucleic acid may comprise a tag. The tag is a molecule or moiety that can be detected, either directly or indirectly. In some embodiments, the tag is an enzyme (e.g., horseradish peroxidase, or HRP), an affinity tag (e.g., streptavidin or biotin), a single-stranded oligonucleotide, a halogen, or any combination thereof conjugated to the reporter nucleic acid. In select embodiments, the tag is a single-stranded oligonucleotide. In other embodiments, the tag is an enzyme. The nature of the tag will dictate the nature of the detection.

The reporter nucleic acid, the guide RNA, the RNA switch, or any combination thereof may be conjugated to a surface (e.g., particle or chip). In some embodiments, the reporter nucleic acid, the guide RNA or the RNA switch are conjugated to a microparticle. In some embodiments, the reporter nucleic acid, the guide RNA or the RNA switch are conjugated to the same microparticle. In some embodiments, the guide RNA and the RNA switch arc conjugated to different microparticles, each microparticle also containing a reporter nucleic acid. In some embodiment, the reporter nucleic acid is conjugated to the microparticle with a single-stranded nucleic acid linker. In some embodiments, the single-stranded nucleic acid linker comprises DNA. In some embodiments, the single-stranded nucleic acid linker comprises RNA.

As used herein, the term “microparticle” refers to small particles having a diameter less than 1 mm. Microparticles may include nanoparticles, those particles with a diameter less than 1 μm. Most microparticles having a spherical, near spherical or spheroidal shape, however, microparticles may also be in the form of rods, chains, stars, flowers, reefs, whiskers, fibers, boxes, and the like. Microparticles may comprise any material, including metals, semiconductor materials, magnetic materials, and combinations of materials. Conjugate chemistries for conjugate the nucleic acid to a variety of microparticles are known in the art and can be employed with the present system.

In some embodiments, the system further comprises a target nucleic acid. In some embodiments, the target nucleic acid comprises the target nucleic acid sequence to which a guide sequence (e.g., a guide RNA) is designed to have complementarity, wherein hybridization between the target nucleic acid sequence and a guide sequence promotes the formation of a complex with a CRISPR RNA-guided Cas-transposase system, or a Cas protein selected from Cas 12a, Cas 13, provided sufficient conditions for binding exist. The target nucleic acid may be DNA or RNA and may be single-stranded or double-stranded. In some embodiments, the target nucleic acid is double-stranded DNA. In some embodiments, the target nucleic acid is single-stranded RNA.

The target nucleic acid may be provided in a sample, e.g., a biological or environmental sample. In some embodiments, the target nucleic acid is provided in a biological sample. The biological sample can be any suitable sample obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces. nasal fluids, and the like. In some embodiments, the sample is blood or blood products. Blood products are any therapeutic substance prepared from human blood. This includes whole blood; blood components (e.g., red blood cell concentrates or suspensions; platelets produced from whole blood or via apheresis: plasma; serum and cryoprecipitate); and plasma derivatives (e.g., coagulation factor concentrates).

The sample can be obtained from the subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

In some embodiments, the target nucleic acid is a nucleic acid from or derived from an infectious agent. The infectious agent may include viruses (e.g., DNA viruses and RNA viruses) and bacteria. The infectious agent may comprise respiratory and vector borne agents.

The system may further comprise a recipient nucleic acid. In some embodiments, the recipient nucleic acid in a double-stranded DNA. The recipient nucleic acid is configured to receive the reporter nucleic acid as a result of a transposition. In some embodiments, the recipient nucleic acid comprises the target nucleic acid sequence to which a guide sequence (e.g., a guide RNA) is designed to have complementarity, wherein hybridization between the target nucleic acid sequence and a guide sequence promotes the formation of a complex with a CRISPR RNA-guided Cas-transposase system, or a Cas protein selected from Cas 12a. Cas 13, provided sufficient conditions for binding exist.

The Cas 12a or Cas 13 enzyme can bind to the tethered gRNAs bound to a target nucleic acid due to the complementarity with the gRNA target nucleic acid sequence. Once bound, the Cas 12a or Cas 13 enzyme cleaves neighboring single-stranded nucleic acid linkers of reporter nucleic acids in close proximity on the same microparticle thereby releasing the tagged reporter nucleic acid sequences into the surrounding solution. In some embodiments, the RNA-guided transposase binds to the tethered gRNAs or a gRNA exposed upon the RNA switch binding to a target singled-stranded nucleic acid and catalyzes transposition (“cut and paste” or “copy and paste” transposition) of a tagged reporter nucleic acid sequence into a target double stranded nucleic acid or a recipient nucleic acid, for example when the target nucleic acid is a singled-stranded nucleic acid. As a result of either mechanism the tagged reporter nucleic acid sequences are released from the microparticle and available for detection in the surrounding solution.

The system may further comprise an indicator nucleic acid. The indicator nucleic acid may be used for recognition of the reporter nucleic acid or the single-stranded oligonucleotide tag. In some embodiments, at least a portion of the reporter nucleic acid is complementary to at least a portion of the indicator nucleic acid. In some embodiments, at least a portion of the single-stranded oligonucleotide tag is complementary to at least a portion of the indicator nucleic acid. The indicator nucleic acid may be immobilized on a solid surface (e.g., chip, plate, membrane, or the like) to immobilize the reporter nucleic acid for detection.

The system may further comprise a linear flow assay (LFA) strip. In lateral flow assays, the liquid sample moves through a matrix or material by lateral flow or capillary action; oftentimes referred to a LFA strip or a test strip. Usually, the sample is applied at the base, or a first end, of the matrix and then the sample moves through that so-called “sample application zone” to a detection zone, which comprises regions have detection agents.

In some embodiments, the LFA strip may comprise a membrane and a sample filter pad on top of a portion of the membrane at first end or the sample application zone. The sample filter pad may be comprised of pores smaller than those of the microparticle, such that microparticles do not pass through the filter pad to the membrane at the first end. The detection zone encompasses the remainder of the membrane and comprises regions having agents which bind and/or detect different tagged reporter nucleic acids, thus allowing detection of a plurality of target nucleic acids on a single strip. For example, an LFA strip may contain a plurality of regions each with different immobilized indicator nucleic acids for detection of a corresponding reporter nucleic acid, or single-stranded oligonucicotide tag on a reporter nucleic acid, as described above. In some embodiments, the LFA strip may contain affinity tags for the tagged reporter nucleic acid to immobilize the reporter nucleic acid and facilitate detection of the tag. The nature of the detection depends on the nature of the tag, colorimetric, spectrophotometric, immunoassay, etc.

The system may further comprise multiplexor chip. Similar to the LFA strip, the multiplexor chip may contain regions comprising agents which bind and/or detect different tagged reporter nucleic acid, thus enabling detection of many target nucleic acids simultaneously on the same chip. The multiplexor chip may have many different sample application zones, each with its own detection zone configured to detect a variety of target nucleic acids. In some embodiments, indicator nucleic acids are conjugated to the multiplexor chip. In some embodiments, affinity tags for the tagged reporter nucleic acid are immobilized on the chip. In some embodiments, the reporter DNA, the guide RNA, the RNA switch, or any combination thereof are conjugated to the multiplexor chip.

The multiplexor may be a field-effect transistor (FET) sensor or other electronic or label-free binding detection systems. In some embodiments, a field effect transistor biosensor may be used and included in the system with the multiplexor. The field effect transistor sensor is a sensing device which can monitor the presence of absence of charged molecules (nucleic acids), ions and the like on a semiconductor material and respond in the form of an electric signal. A variety of FET sensors are known in the art and may be compatible with the present system. FET sensors allow high sensitivity, high selectivity, and real-time monitoring. In some embodiments, the multiplexor is a single-molecule all-electronic detection platform based on a single-molecule field-effect transistor (smFET). The smFET platform may use a field-effect transistor sensor where a single molecular probe (reporter nucleic acid) is attached to a point defect in a single carbon nanotube and the release of the reporter nucleic acid is detected by changes in current levels.

3. Methods of Detecting a Nucleic Acid

The present disclosure provides methods for detection of nucleic acids. The methods may comprise obtaining a sample comprising a target nucleic acid., incubating the sample with: a reporter nucleic acid comprising a tag, at least one or both of: a guide RNA that comprises a nucleic acid sequence complementary to a target DNA sequence and an RNA switch, and a CRISPR RNA-guided Cas-transposase system, or a Cas protein selected from Cas 12a, Cas 13, and combinations thereof to form a sample mixture. In some embodiments, the reporter nucleic acid, the guide RNA, the RNA switch, or any combination thereof are conjugated to a microparticle. In some embodiments, the reporter nucleic acid, the guide RNA, the RNA switch, or any combination thereof are conjugated to the multiplexor chip.

The methods may further comprise incubating the sample with a recipient nucleic acid. In some embodiments, the recipient nucleic acid nucleic acid is configured to receive the reporter nucleic acid as a result of a transposition. The methods may further comprise incubating the sample with an indicator nucleic acid. In some embodiments, the indicator nucleic acid is conjugated to the multiplexor chip or isolated within the linear flow assay strip.

In some embodiments, the methods comprise measuring the presence of the reporter nucleic acid on a LFA strip. In some embodiments, the methods comprise measuring the presence and/or absence of the reporter nucleic acid on a multiplexor chip.

The descriptions and embodiments provided above for the system components (gRNA, RNA switch, CRISPR RNA-guided Cas-transposase system, Cas12a, Cas13, LFA strip, multiplexor chip, target nucleic acid, recipient nucleic acid, reporter nucleic acid, and tag) are applicable to the methods for detecting a nucleic acid.

### 4. EXAMPLES

### Example 1

FET (Fluidic Enzymatic/Electronic Tag-based) Detection

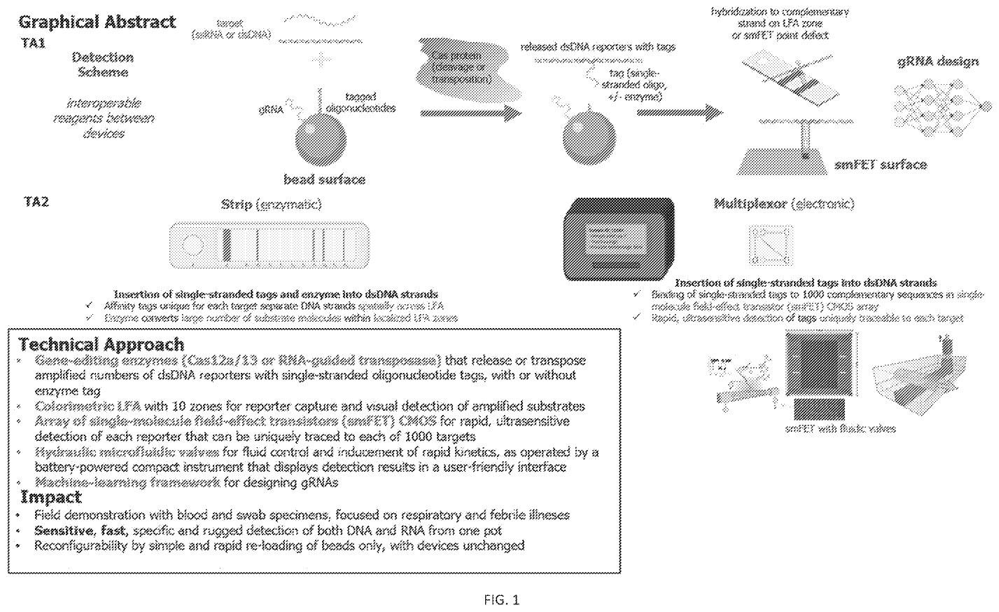

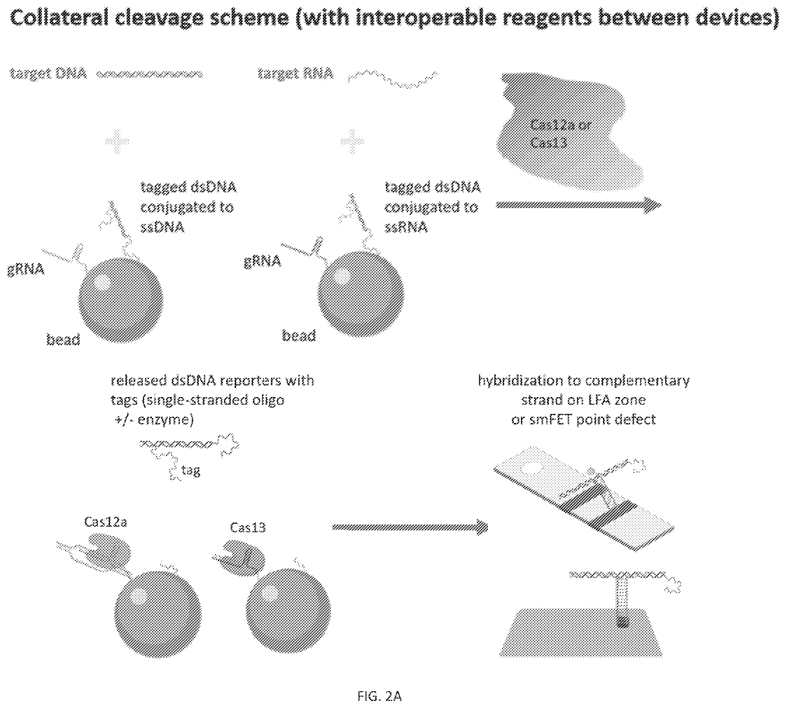

Upon recognition of dsDNA or ssRNA genomic targets, as directed by gRNAs tethered to bead surfaces, the gene-editing enzymes generate tagged reporter sequences in solution (FIG. 1). This generation can take place via using one of two gene-editing methods or systems (FIG. 2).

Cas 12a/Cas 13 enzymes, which bind to tethered gRNAs and localize to bead surfaces, cleave neighboring reporter sequences that are located on the same bead and that are within the gyration radius of the tethered gRNA. In the dsDNA reporter, one of the strands is synthetically conjugated to either ssDNA (for Cas 12a) or ssRNA (for Cas 13). Upon target recognition, activation of Cas enzymes results in collateral cleavage of either the ssDNA or ssRNA linker sequence and releasing the tagged dsDNA reporter sequences into solution.

The RNA-guided transposase, which binds to tethered gRNAs and localize to bead surfaces. Target RNA binds to a designed gRNA “switch” to expose a previously hidden gRNA sequence, which can now also bind the transposase. The transposase recognizes a dsDNA sequence either genomic dsDNA sequence, or in the case of target RNA, a synthetic dsDNA sequence that act as a recipient as complementary to gRNA, and cuts and inserts a neighboring tagged donor DNA into the dsDNA strand. The result is dsDNA reporter sequences, transposed with tagged sequences, in solution.

Either gene-editing system or method can generate tagged dsDNA sequences for downstream detection. The dsDNA reporter sequences contain two types of tags: single-stranded oligonucleotides or enzymes.

For single-stranded oligonucleotides, each oligonucleotide sequence allows unique tracing back to the bead on which a specific gRNA was co-localized, and hence a specific dsDNA or ssRNA target sequence. These oligonucleotides are either ssDNA (for Cas13a and transposase) or ssRNA (for Cas 12, to avoid collateral cleavage of the tags, which could occur if they were ssDNA). The single-stranded oligonucleotides serve an additional important function in selectively hybridizing to complementary ssDNA sequences localized to LFA zones or smFET point defects, ssRNA binds to complementary ssDNA effectively and specifically.

Enzymes are used with an LFA device only. For the FET Strip LFA, an enzyme tag (e.g., horseradish peroxidase, or HRP) is also added.

The detection is sensitive and fast. There is already an initial amplification with the collateral cleavage or repeated transposition of many neighboring tethered reporter sequences (available as a “cloud” of substrate molecules for the Cas enzymes around the gyration radius of the tethered gRNA). The large number of released tagged reporter sequences are then detected via sensitive devices. For the LFA, the enzyme produces a large number of colorimetric substrate molecules within confined zones. For the smFET CMOS array, binding of single complementary oligonucicotide targets can be detected within minutes (this ultrasensitive detection is also quantifiable). No denaturation or pre-amplification of target strands is required. Genomic targets made of dsDNA and ssRNA can be detected from one pot together.

Upon choosing a new panel of targets, only the beads have to be re-loaded with new unique gRNA sequences matched to a pre-defined single-stranded oligonucleotide tag sequences. Neither the LFA strip or the smFET array would need to be modified, as they are already functionalized with the same pre-defined 10 or 1000 ssDNA sequences that are complementary to the single-stranded oligonucleotide tag sequences.

DoD-relevant disease panels. A wide range of RNA and DNA viruses as well as bacterial species that pose risk of illnesses in U.S. and worldwide (Table 1) are used including but not limited to respiratory illnesses and vector borne illnesses. Non-influenza respiratory viruses can cause influenza-like illness. Several respiratory pathogens are easily transferable between humans and animals (e.g., zoonotic) and can cause severe outbreaks such as severe acute respiratory syndrome (SARS), middle east respiratory syndrome (MERS) and influenza pandemics. The vector borne illnesses Dengue, West Nile, Zika and chikungunya viruses are the most common arboviruses spread by mosquito vectors that cause febrile illnesses. Dengue and Zika viruses can cause severe illnesses and lead to fatality. Ticks can cause Powassan fever and Lyme disease. As repeated infections of dengue can progress into severe dengue and dengue hemorrhagic fever, 20 genes that were recently discovered with potential for host response-based prognosis are included.

Over the last 30 years, thousands of human and animal samples from worldwide were collected, covering 20 respiratory and 30 vector borne agents. Here, agents which have ample specimens, previous assays were selected. Since tissue-cultured viruses and related clinical samples are available (Table 1), test samples with contrived and spike-in samples are readily available. Prospective samples (sample and swab) are collected from different sites, such as flu season sample and tick and mosquito collections. Assay performance is regularly assessed against qPCR and other gold standards.

TABLE 1
DoD-relevant targets.

| | Disease | Taxonomic | | | Gene | |
|---|---|---|---|---|---|---|
| Pathogen | type | group | Genome | Probe information | target | Target selection |
| Zika virus | Mosquito- | Flaviviridae | ssRNA | Probe target 1 (20-30 nt) | NS3, | Conserved region for widely |
| [arbovirus] | borne | | | Detects Zika and WNV both, | 3′ UTR | used screening assays |
| | Illness | | | Probe 2 (20-30 nt, ZIKV | | |
| West Nile | Vector | Flaviviruses | ssRNA | only), Probe target 3 | | |
| virus | borne | | Virus | (20-30 nt, WNV only) | | |
| [arbovirus] | Illness | | | | | |
| Chikungunya | Vector | Alphavirus | ssRNA | Probe target 4 (Detects | NS2B | Conserved gene region in West |
| virus | borne | | Virus | all CHIKV) | | African, Asian, and |
| [arbovirus] | Illness | | | | | East/Central/South African |
| | | | | | | (ECSA) lineage |
| Adenoviruses | Respiratory | Adenovirus | dsDNA | Probe target 5 (50-60 nt) | Hexon | CDC recommended assay in |
| | Illness | | | Detects all Human AdV | | Hexon gene |
| Powassan | Tick-borne | Flaviviruses | ssRNA | Probe target 6 (Detects | NS5 | Widely used assay as NYSDOH |
| virus | Illness | | Virus | all POWV) | | is based on conserved region of |
| [arbovirus] | | | | | | NS5 gene |
| Influenza | Respiratory | Orthomyxo- | ssRNA | Probe target 7 (Detects | Matrix | Matrix-based PCR assays are |
| viruses A | Illness | virus | Virus | all Influenza A in USA) | | recommended by WHO to detect |
| Influenza | Respiratory | Orthomyxo- | ssRNA | Probe target 8 (Detects | Matrix | seasonal Influenza detection and |
| viruses A | Illness | virus | Virus | all Influenza B in USA) | | discrimination between strains |
| Borrelia | Vector | Spirochaetaceae | dsDNA | Probe target 9 (50-100 | OsP A | CII in-house assay based on |
| burgdorferi | borne | | | nt) Detects all Borrellia | | ospA gene and detects all isolates |
| [bacteria] | Illness | | | burgdorferi In USA | | from the USA |
| Rickettsia | Vector | Rickettsiaceae | dsDNA | Probe target 10 (50-100 nt) | 17 kDa | Multiple published studies |
| rickettsii | borne | | Bacteria | Detects all Rickettsia | | validated conserved regions in |
| [bacteria] | Illness | | | rickettsiiIn USA | | the 17 kDA gene for molecular |
| | | | | | | assay development |

Gene targets for 10-plea. Probes for the 10-plex FET Strip are selected based on the most conserved gene regions of the pathogenic agents, and where published data are available from qPCR and screening PCR assays. In an exemplary panel, 10 targets that cover different disease types (respiratory and vector-borne) and viral as well as bacterial pathogens, representing RNA and DNA genomes (Table 1), were selected. Banked specimens (clinical and cultured isolates) are readily available. Targets of 20-30 nt for RNA and 50-100 bp for DNA genomes were selected. As emerging pathogens appear, similar analysis is performed to choose an initial 10-plex panel. Gene targets for 1000-plex. The methods target unique sequences that can identify subtype, rare mutations, and host biomarkers of disease severity. Details for target selection based on infectious-disease considerations, followed by an in silico algorithm (first based on pre-existing data, and subsequently developed into a fine-tuned deep-learning neural network based on a high-throughput experimental screen) are explained herein. An exemplary panel is shown in Table 2.

TABLE 2
1000 gene targets for Multiplexor

| | | |
|---|---|---|
| | | Minimum |
| | | Target |
| Family | Genome | Probes |

Respiratory Illness Panel

| | | | |
|---|---|---|---|
| Viruses | | | |
| Human Adenoviruses | Adenoviridae | DNA | 10 |
| Coronavirus HKU1 | Coronaviridae | RNA | 10 |
| Coronavirus NL63 | Coronaviridae | RNA | 10 |
| Coronavirus 229E | Coronaviridae | RNA | 10 |
| Coronavirus OC43 | Coronaviridae | RNA | 10 |
| Human Metapneumovirus | Pneumoviridae | RNA | 10 |
| Human Enterovirus A | Picornaviridae | RNA | 20 |
| Human Enterovirus B | Picornaviridae | RNA | 20 |
| Human Enterovirus C | Picornaviridae | RNA | 20 |
| Human Enterovirus D | Picornaviridae | RNA | 20 |
| Human Rhinovirus | Picornaviridae | RNA | 20 |
| Influenza A | Orthomyxoviridae | RNA | 20 |
| Influenza A/H1 | Orthomyxoviridae | RNA | 20 |
| Influenza A/H3 | Orthomyxoviridae | RNA | 20 |
| Influenza A/H1-2009 | Orthomyxoviridae | RNA | 20 |
| Influenza B | Orthomyxoviridae | RNA | 20 |
| Parainfluenza Virus 1 | Paramyxoviridae | RNA | 10 |
| Parainfluenza Virus 2 | Paramyxoviridae | RNA | 10 |
| Parainfluenza Virus 3 | Paramyxoviridae | RNA | 10 |
| Parainfluenza Virus 4 | Paramyxoviridae | RNA | 10 |
| Respiratory Syncytial Virus A | Pneumoviridae | RNA | 10 |
| Respiratory Syncytial Virus B | Pneumoviridae | RNA | 10 |
| Bacteria | | | |
| Bordetella parapertussis | Alcaligenaceae | DNA | 10 |
| Bordetella pertussis | Alcaligenaceae | DNA | 10 |
| Chlamydia pneumoniae | Chalmydiaceae | DNA | 10 |
| Mycoplasma pneumoniae | Mycoplasmataceae | DNA | 10 |

Vector Borne Illness Panel

| | | | |
|---|---|---|---|
| Viruses | | | |
| Chikungunya virus | Togaviridae | RNA | 10 |
| Dengue virus 1 | Flaviviridae | RNA | 20 |
| Dengue virus 2 | Flaviviridae | RNA | 20 |
| Dengue virus 3 | Flaviviridae | RNA | 20 |
| Dengue virus 4 | Flaviviridae | RNA | 20 |
| West Nile virus | Flaviviridae | RNA | 10 |
| Yellow Fever virus | Flaviviridae | RNA | 10 |
| Powassan virus | Flaviviridae | RNA | 10 |
| Zika virus | Flaviviridae | RNA | 10 |
| Bacteria | | | |
| Borrelia burgdorferi | Spirochaetaceae | DNA | 10 |
| Rickettsia rickettsii | Rickettsiaceae | DNA | 10 |

Host severity. As repeated infections of dengue can progress into severe dengue and dengue hemorrhagic fever, 20 genes that were recently discovered with potential for host response-based prognosis are included. Beyond dengue, host gene expression biomarkers assess disease severity and prognosis (˜200 probes; see Table 3) in blood. These ssRNA markers including circulating microRNAs, and 4 markers as identified by RNA sequencing of whole blood samples indicative of a general host systemic response to many types of viral infection. If needed, protein markers such as chemokines, C-reactive protein and procalcitonin can be detected in the smFET array platform by attaching antibodies to the point defects.

TABLE 3
200 nucleic-acid targets of host severity to
dengue as well as broad viral infections.

| | | |
|---|---|---|
| | Minimum | |
| | Target | |
| Gene Name | Probes | Reference |

20-Gene Set Predictive of Progression to Severe
Dengue (and possibly other Flaviviruses)

| | | |
|---|---|---|
| Defensin Alpha 4 | 5 | PMID: |
| Glycogenin 1 | 5 | 30699342 |
| Torsin Family 2 Member A | 5 | |
| Protein Tyrosine Phosphatase, Receptor Type M | 5 | |
| Spondin 2 | 5 | |
| GRB2-Related Adaptor Protein 2 | 5 | |
| Calcium Voltage-Gated Channel Auxiliary | 5 | |
| Subunit Alpha2 delta 3 | | |
| Calcium Voltage-Gated Channel Auxiliary | 5 | |
| Subunit Alpha2 delta 3 | | |
| Transmembrane Protein 63C | 5 | |
| Adenylate Kinase 5 | 5 | |
| Chromodomain Helicase DNA Binding Protein 3 | 5 | |
| C-X3-C Motif Chemokine Receptor 1 | 5 | |
| Transcriptional Regulating Factor 1 | 5 | |
| Guanylate Binding Protein 2 | 5 | |
| Serine Incorporator 5 | 5 | |
| SRY-Box 13 | 5 | |
| Natural Cytotoxicity Triggering Receptor 3 | 5 | |
| ABI Family Member 3 | 5 | |
| Chromosome 3 Open Reading Frame 1 | 5 | |
| Ectonucleotide Pyrophosphatase/ | 5 | |
| Phosphodiesterase 5 | | |

Other DNA/RNA Severity Biomarkers

| | | |
|---|---|---|
| Circulating microRNAs as Potential Biomarkers | 50 | PMID: |
| of Infectious Disease | | 28261201 |
| Interferon Stimulated Gene 13 (ISIG15) | 10 | PMID: |
| Interleukin 16 (IL16) | 10 | 28588308 |
| 2′,5′-Oligoadenylate Synthetase Like (OASL) | 10 | |
| Adhesion G Protein Coupled Receptor E5 | 10 | |
| (ADGRE5) | | |

### Example 2

Biochemical Reconstitution of Enzymatic Activity

To develop molecular detection technologies based on target-activated collateral cleavage activity, candidate CRISPR-Cas12 and Cas13 orthologs are generated following previously published protocols. In brief, proteins are over-expressed in E. coli and purified using standard approaches. Guide RNAs (gRNAs; also, often referred to as crRNAs) are generated by in vitro transcription using T7 RNA polymerase, which allows for rapid and scalable screening. Alternatively, gRNAs are generated by chemical synthesis or outsourced to commercial vendors, for example, when subsequent bioconjugation steps are used. The quality and enzymatic activity of Cas 12 and Cas 13 orthologs are benchmarked by comparing the reagents to previously published data for validated gRNA-target pairs, using cleavage of fluorophore-quencher pairs as a sensitive and robust fluorescence-based readout. Although target amplification steps may be performed for benchmarking purposes, for example to compare the reagents head-to-head with SHERLOCK and DETECTR technologies which require amplification, the steps are not required or used in the FET Strip and FET Multiplexor assays due to other fast signal-amplification processes in the scheme.

High-throughput testing of disease-relevant gRNA-target pairs. Using the fluorescence-based readout described above, large panels of candidate gRNAs and their associated targets (either RNA or DNA, depending on the pathogen) are biochemically profiled in 384-well plates. After ensuring that short model target substrates activate robust collateral cleavage, native genomic RNA/DNA substrates, as well as spike competitor RNA/DNA into reactions, are isolated and tested to ensure that detection is not compromised in more complex nucleic acid mixtures. Exhaustive screening for both false negatives. e.g., gRNAs that are unable to guide sensitive detection of a target sequence of interest, and false positives, e.g., gRNAs that promiscuously elicit a fluorescent signal in the absence of a cognate target, is used. To avoid false positives, specificity is rigorously assessed both by screening validated gRNAs against a large panel of “Off-target” (non-matching) sequences and samples, and also by synthesizing partially matching gRNAs and ensuring that they can accurately discriminate the target molecule. Large experimental datasets that have been recently generated for candidate Cas12 and Cas13 orthologs, are used to aid in gRNA design. Larger-scale gRNA screening is likely feasible in a cell-based assay, however, results from in vivo specificity profiling will inevitably differ from in vitro results, as the gRNA stability, target search kinetics, and target-activated collateral cleavage activity will differ between these conditions. Therefore, parameter screening is focused on biochemical conditions (including storage conditions, reaction mixture pH and salts, and assay temperature) that are highly adapted to the devices used herein.

Evaluation of biochemical stability after prolonged storage, and in biofluids. As stability of all protein and gRNA reagents is important, biochemical activity is assessed after various storage protocols, upon resuspension/rehydration in a compatible aqueous reaction buffer. Previous work demonstrated functional target detection after a round of lyophilization and rehydration for Cas 13-gRNA complexes, those, and further additional storage conditions (e.g., in lyophilized states for enzymes stored in Mastermix, and bead-tethered gRNAs and DNA-protein conjugate reporter sequences) are tested for the reagents. Additionally, relying on a fluorescence-based readout, the detection efficiency and sensitivity when a target sequence is present within relevant biofluid mixtures, including blood and swab, is systematically tested. The fast assay time (<15 min) likely outcompetes low-level degradation of protein-gRNA reagents. Nevertheless, proteases and nucleases in biofluids may impose a limit on sensitivity, and different inhibitors and stabilizers are tested using the same high-throughput fluorescence assay.

### Example 3

Biochemical reconstitution of RNA-guided transposase activity

An RNA-guided transposase system is described in which Cas proteins (e.g., Cascade or Cas12k) together with transposase proteins (e.g., TnsB and TnsC, sometimes with TnsA) mediate targeted DNA integration downstream of genomic dsDNA sites complementary to gRNA. In one embodiment, the system derives from a Vibrio cholerae transposon, and relies on a complex known as QCascade for RNA-guided DNA recognition, and a heteromeric, catalytically active transposase known as TnsABC, for integration of donor DNA within the targeted DNA molecule (See U.S. Patent application Ser. No. 16/812,138, incorporated herein by reference in its entirety). Notably, this transposition reaction, which is analogous to that of the Tn7 transposon which has been the focus of more than two decades of genetic and biochemical characterization, involves concerted DNA cleavage and transesterification reactions, whereby the mobile element is excised from its donor source, and inserted within the target. This chemistry is used to liberate reporter DNA molecules immobilizes on a solid support, and insert them into the target dsDNA upon recognition, for downstream detection.

Like canonical CRISPR-Cas systems, CRISPR-transposon systems are remarkably diverse, and bioinformatics analyses indicate that Tn7-like transposons have coopted at least three different types of CRISPR-Cas machinery. Zhang and colleagues described RNA-guided transposases that also employ TnsBC, but which are guided by an alternative RNA-guided DNA targeting complex Cas12k. Cas12k is a distant, non-cutting homolog of Cas12a, which has native inactivating mutations in the RuvC nuclease domain. Multiple orthologous RNA-guided transposases falling within each subfamily in an E. coli-based transposition assay, may also be used to optimize optimizing both efficiency and specificity. The V. cholerae CRISPR-transposon has efficiencies routinely maxing out at 100% (without selection) and on-target specificities>95% across dozens of gRNAs; in stark contrast to the ShCAST system described previously, which showed rampant off-target integration and low gRNA success rates.

QCascade was previously purified and high-resolution structures were determined by cryo-electron microscopy (cryoEM). More recently, the remaining components (TnsABC) were purified and cryoEM structures of TnsC were determined. Biochemically reconstituted RNA-guided DNA integration from purified components allows measurement of the specificity, transposition kinetics, turnover, donor DNA permissiveness, and multiplex capabilities of the reconstituted RNA-guided transposase system. Previously validated transposition assays involving radiolabeled donor DNA and target DNA substrates, which allow for sensitive tracking of all bond breakage and joining events, are used. Critical controls include both non-targeting and partially mismatched gRNAs, as well as inactive donor DNA substrates lacking the necessary sequence elements required for excision. Next-generation sequencing (NGS) determines the exact nucleotide junctions formed upon successful RNA-guided DNA integration.

High-throughput testing of disease-relevant gRNA-target pairs. The NGS approach is used to systematically investigate mismatch sensitivity of RNA-guided DNA integration in a large, pooled library experiment. Specifically, oligonucleotide array synthesis is employed to generate target DNA sequences containing large libraries of defined, off-target sequences, and then reacts RNA-guided transposase components with donor DNA and the library of target DNAs. Using an NGS approach similar previous work which selectively amplifies only target DNA molecules containing the inserted donor DNA, deep sequencing of the products is used to computationally determine the specificity profile for a given gRNA, gRNAs produced by the algorithms described below may be used. The same analyses are performed in parallel reactions with additional gRNAs, to systematically profile the set of disease-relevant gRNAs.

Evaluation of biochemical stability after prolonged storage, and in biofluids. A similar panel of storage, buffer, and inhibitor/stabilizer conditions as described above for Cas 12 and Cas 13, is explored and used to determine a parameter space where RNA-guided DNA integration efficiencies are maximized.

### Example 4

Generation of Donor Reporters and Tags for Transposition

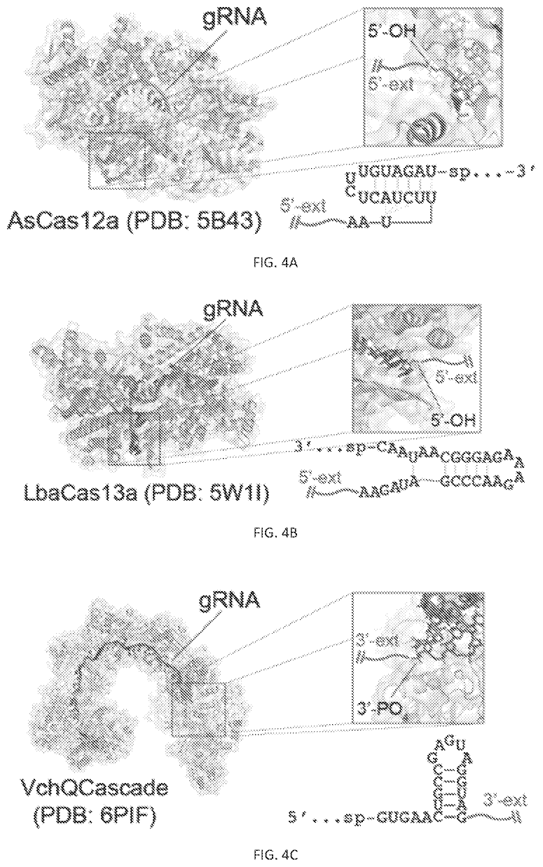

The devices have gRNA and reporter nucleic acids tethered to bead surfaces. A structure-guided approach is used to engineer extensions onto the gRNAs for the detection platforms, in a manner that enables immobilization but has no adverse effect on targeting or collateral cleavage (FIG. 4).

Cas 12a and Cas 13 both recognize gRNAs containing invariant repeat-derived sequences at the 5′ end of the gRNA, upstream of the ‘spacer’ sequence that hybridizes to a target nucleic acid. Multiple high-resolution structures have been determined for both enzymes, highlighting the mechanism of gRNA scaffold recognition, and revealing an accessible 5′ terminus amenable to linker extension. Importantly, both enzyme families also possess a precursor CRISPR RNA ribonuclease domain, which in native CRISPR-Cas systems, allows for processing of long transcripts derived from CRISPR arrays into mature gRNAs. These ribonuclease domains are completely independent of the RuvC and HEPN domains that cleave target DNA and RNA for Cas12 and Cas13, respectively; furthermore, the active sites for both ribonuclease domains have not only been identified, but specifically inactivated with point mutations, without any adverse effects on catalytic activity of the RuvC/HEPN domains in cleaving nucleic acid targets. Therefore, the generation of variant Cas 12 and Cas 13 nucleases that possess wild-type activity for target cleavage (and thus, detection), but are unable to enzymatically process modified gRNAs containing 5′ extensions, is readily accessible. These variants are designed purified and biochemically profiled using the activity assays outlined above and used in the experiments described below.

5′-extended gRNAs for Cas 12 and Cas13 arc generated and the efficiency, sensitivity, and specificity of target detection is compared using the fluorescence-based assay described above. The 5′ ends of these extended gRNAs are derivatized, such that they can be covalently tethered to a solid support. These gRNAs are immobilized to micron-sized beads, and the same activity assays are performed in order to compare detection parameters with Cas12-gRNA and Cas13-gRNA that are freely diffusing in solution versus tethered to a solid support. In the course of these experiments, a variety of linker sequences, linker lengths, and chemistries are used, while optimizing for maximal detection sensitivity.

For RNA-guided transposases, the gRNA extension strategy varies, depending on the system. For Cas12k-directed transposases, similar 5′ extensions as described for Cas12a are employed. For QCascade-directed transposes, 3′ extensions to the gRNA are used, as the 5′ end of the gRNA is buried within the Cas8 subunit. In an analogous manner to Cas 12 and Cas 13, QCascade possess natural ribonuclease activity in the Cas6 subunit that is essential for precursor RNA processing in native CRISPR-Cas systems. However, this ribonuclease domain can also be inactivated with a simple point mutation, and high-resolution structures indicate that the 3′ terminus of the gRNA within QCascade is solvent exposed and accessible to tagging. Using the biochemical activity assays described above for RNA-guided transposases, the DNA integration activity is compared for QCascade variants containing Cas6-inactivating mutations as well as 3′-extended gRNAs. Extended gRNAs arc also conjugated to micron-sized beads using the same chemistry to investigate whether DNA excision and integration is impacted.

### Example 5

Design and Functional Testing of Surface-Tethered Reporter Probes

Tagged reporter molecules that are compatible with both devices are designed and tested. For the collateral cleavage activities of Cas 12 and Cas 13, reporter probes based on recent studies that systematically assess each homolog's sequence requirements are designed. These probes are conjugated with enzymatic reporters and single-stranded oligonucleotide tags, as well as chemical modifiers to enable surface immobilization. An assay quantifies successful release of the reporter from a solid support, using qPCR on the reporter sequences and/or ELISA on a HRP enzyme tag. The stability of surface-immobilized reporter probes in the presence of biofluids is also evaluated.

RNA-guided transposases recognize conserved sequences at the left and right ends of donor DNA but can cut-and-paste natural or synthetic payloads ranging from 102-105 bp in size. Because DNA recognition is limited to only the terminal ˜100 bp at each end, non-DNA moieties are conjugated within the internal payload, for sensitive downstream detection events. Using commercially available chemistries (including biotin dT, amino modifier dT, azide modification, alkynes, 5-octadinynyl dU, or thiol modifiers) or well-established protein-DNA conjugation chemistries, chimeric transposon donor DNA molecules are generated that contain enzymatic reporters (e.g., horseradish peroxidase, or HRP) and affinity tags (e.g., ssDNA, due to straightforward multiplexing, proteins such as glutathione transferase) for use in FET Strip, ssDNA tag lengths are about 30-mers, such that there is little to no chance of rare target sequences in the specimens matching the tag sequences. For use in FET Multiplexor, halogen (e.g., fluorine) additions at the 2′ or 3′ positions in the phosphodiester backbone are used. Any loss of specificity or efficiency for RNA-guided transposases when dsDNA donors are compared with donors with chimeric DNA-reporter conjugates is evaluated.

Conjugation to dsDNA reporter sequences. The tethering of oligonucleotides to the micron-sized beads (e.g., polystyrene) will be through standard chemistries such as thiol linkages. Additional commercially available chemistries include biotin dT, amino modifier dT, azide modification, alkynes, 5-octadinynyl dU, or thiol modifiers) and well-established protein-DNA conjugation chemistries. Chimeric dsDNA molecules are generated that contain enzymatic reporters (e.g., HRP) and affinity tags (initially ssDNA, due to straightforward multiplexing, but, if needed, proteins such as glutathione transferase). No loss of specificity or efficiency for enzymatic activity is verified for surface-tethered chimeric DNA-reporter conjugates compared to substrates in solution.

As an alternative to conjugation internal to the dsDNA sequence, one of the DNA strands is extended to a ssDNA oligonucleotide tagging sequence (e.g., at the opposite terminus from the bead surface).

### Example 5

Computational Design of gRNAs

gRNAs are designed two steps: target sequence selection and gRNA selection. Target sequences from the pathogen or human genome are selected based on the type of probes (broad or specific), from which all legitimate guide sequences will be scored for on-target efficacy and off-target potential. Although gRNAs for RNA-guided transposases differ from other Cas systems (such as Cas9 or Cas13), much is already known about the recognition of dsDNA by Cascade (and Cas12) in terms of seed sequence and mismatch discrimination. To ensure that the design of transposase gRNAs accounts for more subtle determinants (such as positional mono/di-nucleotide preferences and mismatch tolerance), a massively parallel reporter system for assessing transposase activity is generated, and the system is screened using: 1) a pool of gRNAs, and 2) a pool of “hidden gRNA switches” (as described below) with target RNA present. The on-target and off-target enzyme activities are used to train a deep neural network model. In select instances, off-target activation for small mismatches is assessed, as such gRNAs could be used for targeting with reduced specificity, to account for slight pathogen variability; positive FET Detector results from gRNAs with “reduced specificity” are clearly delineated as such in the report to the user.

The deep-learning model incorporates non-sequence features known to be important for other CRISPR systems, such as seed region and secondary structures of gRNAs. The architecture of the neural network is optimized, including the number of neurons for each layer, number of each type of layers, and how they are connected. To incorporate RNA folding and other information, non-sequential models is used. For training and validation, most of the data (80%) is used for training and the rest is used for validation. Once the model is validated, broad and strain-specific targets are chosen from the panels from Table 1, and gRNA sequences are selected. On the fly, with new agents or strains identified, the algorithm may be available on the cloud for input of new target sequences and output gRNA and gRNA switch sequences in less than 5 minutes.

The in silico pipeline selects the gRNA that maximizes orthogonality to other targets in the pot and avoids unintended non-specific activity, while at the same time also maximizes on-target detection sensitivity. The initial gRNA scoring model uses pre-existing empirical rules, high-throughput data, and machine learning models. The model also incorporates the data from experimentally testing a large number of gRNAs. A deep neural network-based model to predict gRNA efficiency and specificity is developed.

Target sequence selection. Broad and specific sequence targets are used.
Broad probes: the genome sequences for all strains/subtypes/isolates of a pathogen of interest are aligned to identify invariable regions. All legitimate guide sequences with a protospacer adjacent motif (PAM) strictly reside in those invariable regions are scored and the optimal gRNA is selected.
Specific probes: from the multiple alignment regions or polymorphisms that are unique to each strain are identified. All legitimate guide sequences overlapping with these unique regions/polymorphisms are scored.

gRNA scoring and selection. For each target sequence selected above, all legitimate guide sequences with a proper PAM are scored. Each gRNA receives an on-target efficacy score and an off-target potential score. The on-target efficacy score prioritizes those gRNAs with potent activity and penalizes those that will causes problems in oligo synthesis or gRNA transcription, folding, and loading into the Cas protein. The off-target potential summarizes the presence of perfect or near-perfect matches in target sequences of other gRNAs in the pot, in the human genome, or other commonly present sequences that are not of interest, such as environmental contaminants. The pipeline output is a ranked list of gRNAs with minimal off target potential, from which a gRNA with the maximum on-target efficacy is chosen.

Generation of strand-displacement “hidden gRNA switches” for direct detection of ssRNA targets. In the FET Detector scheme, detection of dsDNA is direct, because gRNA directs the transposase to bind dsDNA and insert a locally available donor DNA sequence into the dsDNA. For direct detection of ssRNA, two reporter elements are added: hidden gRNA switches (whereby the binding of ssRNA target disrupts a hairpin structure to expose gRNA), and synthetic dsDNA (that act as recipients for transposition). These switches, based on strand-displacement principles, are based on antisense RNA, and on toehold switches for translation and transcription. More directly, for gene editing, recent work has shown the feasibility of such switches for exposing hidden gRNA sequences upon binding of ssRNA targets, where binding of a ssRNA target to a complementary RNA sequence within a hairpin structure results in a structural change that exposes a previously hidden gRNA sequence. With the exposed gRNA, the transposase inserts locally available donor DNA into a “reporter dsDNA”, which is synthetic dsDNA with a sequence recognized by the gRNA, and acts as recipient for transposition.

Development of Basic in Silico Pipelines Using Pre-Existing Data and Models.

Cas3 and RNA-guided transposase: There are currently no systematic studies or in silico models for gRNA on-target efficacy or off-target potential for either the transposase system or the Cas 13a/b systems that have been tested for in vitro RNA detection. A machine learning model recently became available for a different Cas13 protein, RfxCas13d, which has not been tested for in vitro RNA detection. A gRNA scoring model is created by using empirical rules learned from other CRISPR systems. For on-target efficacy, these rules include: 1) avoid guide sequences that form strong structures within the gRNA, which may prevent loading into the Cas protein/complex; 2) avoid homopolymers in the guide sequence that inhibit gRNA expression or interfere with oligo synthesis. For off-target potential, similar sequences (less than 6 mismatches) in the non-targeting sequences are searched, including targets of other gRNAs, the human genome, and other common environmental contaminant sequences in the sample. For RNA targeting, candidate toehold switches are designed using a pre-existing workflow based on NUPACK. The toehold switch oligo is scored similarly for both on-target efficacy and off-target potential, and the scores are added to gRNA scores.

Cas2: Multiple deep neural network-based models have been developed for predicting the on-target efficacy and off-target activity of AsCas12a (AsCpfl) by training with the on-target activity of over 15,000 gRNAs in human cells, as well as genome-wide off-target activity assayed by two different methods. An attention boosted deep neural network model was reported to have the highest performance and is used to score all protospacer sequences with a proper PAM (the code is publicly available on GitHub).

High-throughput measurements of gRNA activities for RNA-guided transposase. To facilitate the development of in silico models for scoring transposase gRNAs, the activity of a large number of gRNAs is measured in vitro. Specifically, a pool of up to 120,000 gRNAs is generated using commercial oligo synthesis followed by in vitro transcription. These gRNAs target sites in the mouse genome that are different between two commonly used strains (C57BL/6J and 129S 1/SvImJ), such that both on-target efficacy and off-target activity of each gRNA is measured simultaneously when both genomic DNA preps are present for targeting. After the in vitro transposition reaction, the insertion site is cloned by using Tn5 tagmentation followed by PCR with a tag primer and a primer within the donor sequence. Illumina sequencing is used to quantify the insertion frequency at each site.

High-throughput measurements of gRNA efficacy and specificity for Cas13. A high-throughput screening assay was previously developed for RfxCas 13d targeting and is used to test the efficacy and specificity of a large number of PsmCas 13b gRNAs.

Development of deep neural network models and an integrated in silica pipeline. Deep neural network models predict gRNA activities for both the transposase system and the Cas13 system. With a hybrid structure, the model extracts both sequence and structure features from the input gRNA and a candidate target (may contain mismatches) to predict targeting efficiency (FIG. 5). The sequence module is similar to a state-of-the-art model for predicting Cas 12a targeting activity, which takes advantage of two of the most successful deep learning architectures: convolutional neural network (CNN) and attention-based transformer. Specifically, the gRNA-target pair is represented as a 3D matrix in the first embedding layer. The output feeds into the second layer, the Transformer, which has shown superior performance on sequential input. The third component consists of multiple CNNs with max-pooling in between. The output is flattened and concatenated with output from the structure module. The structure module computes the secondary structures of the gRNA, the target (RNA target only), and the gRNA-target co-folding by using the ViennaRNA package. The pairing probability for each nucleotide position is represented as vectors and the output will be concatenated with that of the sequence module. A fully connected dense layer outputs the targeting efficiency for the input gRNA-target pair.

The data generated from the high-throughput assays, e.g., the targeting efficiency for each gRNA-target/off-target pairs, is divided into a training set and a test set. The model is trained with cross-validation in the training set, and the test set is used to evaluate the final model.

Once established, the neural network model is integrated into the in silico pipeline for designing gRNAs for each target sequence. On the fly, with new agents or strains identified, the algorithm can be available on the cloud for input of new target sequences and output gRNA sequences and gRNA switch sequences in less than two hours.

Systematic test of the in silico pipeline for new target sequences. New target sequences are generated and the in silico pipeline is used to design gRNAs for those sequences. The efficacy and specificity of those gRNAs is tested experimentally using the in vitro assays.

Development of computer interface for gRNA design. An easy-to-use interface is used for designing gRNA. Users upload a single or multiple target sequences and the software will output the most efficient and specific gRNAs.

Agile, iterative design of new gene targets and gRNAs. As new sequences of emerging pathogens and strains become available, new gene targets arc selected through an iterative process. As an illustration, for dengue serotype DI, 2032 complete genomes were downloaded from ViPR (Virus Pathogen Resource) database. Nucleotide sequences that code (CDS sequences) for NS3, and NS5 genes were trimmed and used to generate target sequences. An iterative sliding window algorithm with a sequence length of 25 nt and offset of 1 nt, was used with the goal to generate targets that cover all 2032 genomes. Six target 25-nt sequences are used to detect all NS3 and NS5 sequences for DENV 1. This strategy leads to generation of additional probes until they cover conserved regions across all whole genomes available in the database. An automated script re evaluates selected probes every six months for coverage of new database entries and triggers an update of selected probes if necessary.

### Example 6

FET Strip

Overall, the user only needs to place the sample into a tube and transfer the solution into an in-line LFA adapter and initiate the test; the total time to answer is 15 minutes (Table 4). First, the user adds 150 μL of the blood or swab sample to a “lysis tube” (for example, Tris-HCl, EDTA, TritonX, and lysozyme, which has been shown to lyse simultaneously Gram-positive and negative bacteria and viruses). Although these lysis components have been shown to be compatible with downstream endonuclease reactions, if needed, an additional 30-second step of passing lysate through a nitrocellulose membrane followed by wash and elution into buffer can be performed. Second, the user transfers the free genomic DNA and RNA into an LFA adapter where further downstream steps such as filtration and reaction with the mastermix are carried out in an automated fashion. PGP-24,T1

TABLE 4
Time to answer for FET Strip.

| | | Time needed |
|---|---|---|
| | Operation | (minutes) |
| 1 | User step 1. In lysis tube: sample lysis | 4 |
| 2 | Nitrocellulose membrane purification (optional) | 0.5 (optional) |
| 3 | User step 2. In LFA adapter: solubilizing freeze- | 0.5 |
| | dried mastermix | |
| 4 | In LFA adapter: reaction with conjugated beads for | 5.5 |
| | oligonucleotide release | |
| 5 | Sample flow through the LFA | 3 |
| 6 | Colorimetric substrate flow through the LFA | 1.5 |
| | Total | 15 |

An automated, in-line sample preparation adapter connects to the inlet end of the LFA. The adapter has a “reaction chamber” that contains RNase inhibitors and:
Mastermix, in freeze-dried form with stabilization reagents, comprising reconstituted Cas-enzymes (and synthetic reporter dsDNA for detecting ssRNA when using the transposition scheme),
Beads of 10 different types (one for each target), with each type containing on its surface:
   one of 10 possible RNA sequences, usually as gRNA (or hidden gRNA switches for detecting ssRNA using the transposition scheme) (FIG. 2).
   one of 10 possible donor DNA sequences, which are conjugated to an enzyme (e.g., HRP) and one of 10 single-stranded oligonucleotide (ssDNA or ssRNA) tags (see FIG. 2 for details). On any one bead type, the combination of a specific oligonucleotide tag and gRNA sequence only appears on that bead type. The attachment of an enzyme, compared to a visible gold nanoparticle or fluorescent tag in traditional LFAs, enhances the signal by several orders of magnitude by turning over thousands to millions of substrate molecules during minutes of incubation. Many copies of the enzyme may be inserted into donor DNA to further multiply the catalytic amplification.

As the RNA is likely the least stable component, the RNA stable® reagent from Biomatrica is used, which stabilized RNA in a dried form on a surface for 22 months at room temperature. Optionally, modified DNA is added to the RNA to increase stability, and the DNA later displaced.

Upon adding the solution to the adapter inlet, the user would press a small bulb, generating positive pressure to push the liquid through a nitrocellulose filter and into the reaction chamber. After a few minutes of reaction, the user presses the bulb again to dispense the solution from the reaction chamber into the LFA inlet. Reporter dsDNA strands contain uniquely identifiable oligonucicotide tags, which can be traced back to gRNA sequences and hence the original dsDNA or ssRNA targets (Table 5).

TABLE 5
Identification of targets in a FET Strip. The strategy will be scaled up to 10 targets.

| | | Bead type that | | | |
|---|---|---|---|---|---|
| | Target | recognizes target | The bead | Reporter | |
| Target | sequence | sequence would | type also | sequence in | |
| number | present | contain . . . | contains . . . | solution | LFA |
| 1 | adenovirus | “gRNA 1” against | reporter | cleavage of ssDNA | positive band |
| (Cas 12a) | dsDNA | target 1 | DNA with | linker to release | in zone 1 |
| | | | HRP and | dsDNA reporter | (which |
| | | | “oligo tag | with HRP and | captures |
| | | | 1” | ssRNA “oligo tag | “oligo tag 1”) |
| | | | | 1” | |
| 2 | influenza | “gRNA 2” against | reporter | cleavage of ssRNA | positive band |
| (Cas 13) | ssRNA | target 2 | DNA with | linker to release | in zone 2 |
| | | | HRP and | dsDNA reporter | (which |
| | | | “oligo tag | with HRP and | captures |
| | | | 2” | ssDNA “oligo tag | “oligo tag 2”) |
| | | | | 2” | |
| 1 | adenovirus | “gRNA 1” against | donor DNA | adenovirus dsDNA | positive band |
| (transposition) | dsDNA | target 1 | with HRP | with HRP and | in zone 1 |
| | | | and “oligo | ssDNA “oligo tag | (which |
| | | | tag 1” | 1” inserted | captures |
| | | | | | “oligo tag 1”) |
| 2 | influenza | “RNA switch” with | donor DNA | synthetic reporter | positive band |
| (transposition) | ssRNA | detection sequence | with HRP | dsDNA with HRP | in zone 2 |
| | | against target 2. | and “oligo | and ssDNA “oligo | (which |
| | | “gRNA 2” on the | tag 2” | tag 2” inserted | captures |
| | | switch is exposed. | | | “oligo tag 2”) |

Because the beads are larger than the pores of the paper, the only oligonucleotide tags (and enzymes) that move through the strip are the ones carried by recipient dsDNA sequences. The LFA contains 10 zones, at <100-um width each, containing complementary ssDNA (for example, micropatterning oligonucleotides) that bind to a specific oligonucleotide tag. Oligonucleotide tags made of ssDNA or ssRNA bind effectively to complementary ssDNA on the LFA. After the sample passes through the 10 zones, the user dips the LFA into a substrate (e.g., 3,3′,5,5′-tetramethylbenzidine for HRP) to initiate catalytic conversion to colored product. The tight localized capture areas in each zone (about two orders of magnitude smaller in area compared to diffuse millimeter-scale spots on an LFA) concentrate the signal to further increase sensitivity. A visible band at each zone corresponds to a known target. As all the targets are exposed to all the recognition sequences within the same tube (e.g., no splitting or compartmentalization of samples), the required sensitivity of ˜10 copies/150 μL and statistics of target recognition will not change significantly upon multiplexing. Currently, the FDA-approved LFA tests from Maxim Biomedical have over 24 months for expiration, with 24-month real time stability and up to 12-month accelerated stability up to 42° C.

### Example 7

FET Multiplexor

TABLE 6
Time to Answer for FET Multiplexor

| | | Time needed |
|---|---|---|
| | Operation | (minutes) |
| 1 | Sample lysis | 4 |
| 2 | Nitrocellulose membrane purification (optional) | 0.5 (optional) |
| 3 | Solubilizing freeze-dried mastermix | 1 |
| 4 | Reaction with conjugated beads for oligonucleotide | 5.5 |
| | release | |
| 5 | Direct detection of DNA hybridization on smFET | 4 |
| | Total | 15 |

Disposable Chips: single-molecule field-effect transistors (smFET), in this effort, existing efforts facilitate developing and bringing to manufacturing a new single-molecule all-electronic detection platform based on the single-molecule field-effect transistor (smFET). Unlike traditional ensemble arrays where hybridization of many molecules is required for signal generation, smFET sensors detect hybridization with a single target molecule via a “point functionalization” of a single carbon nanotube-producing a high signal-to-noise ratio on an isolated sensor, which can determine the concentration of a target by measuring the time between mass-transport-limited capture events. Adaptation of these sensors to large arrays—such as on a complementary metal-oxide-semiconductor (CMOS) platform—leads to a dramatic advance in performance, with many more probe interactions recorded per unit time. It allows the sensors to be manufactured at wafer scale achieving low manufacturing costs. The advantages of smFET—single molecule detection, the low cost of the platform, and the absence of optical hardware—has the potential to impact a wide range of applications.

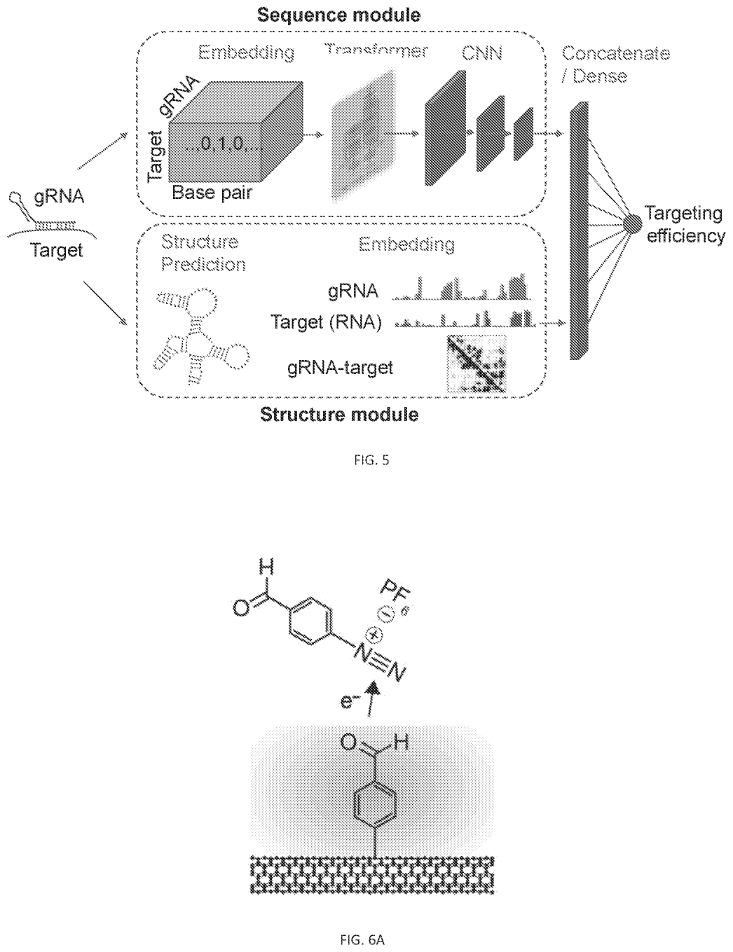

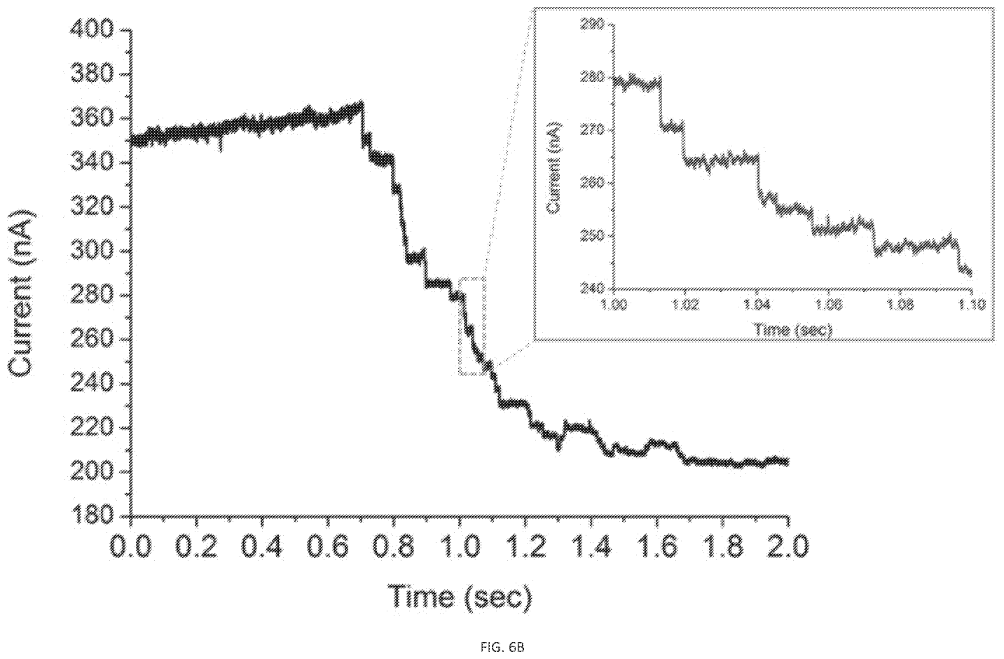

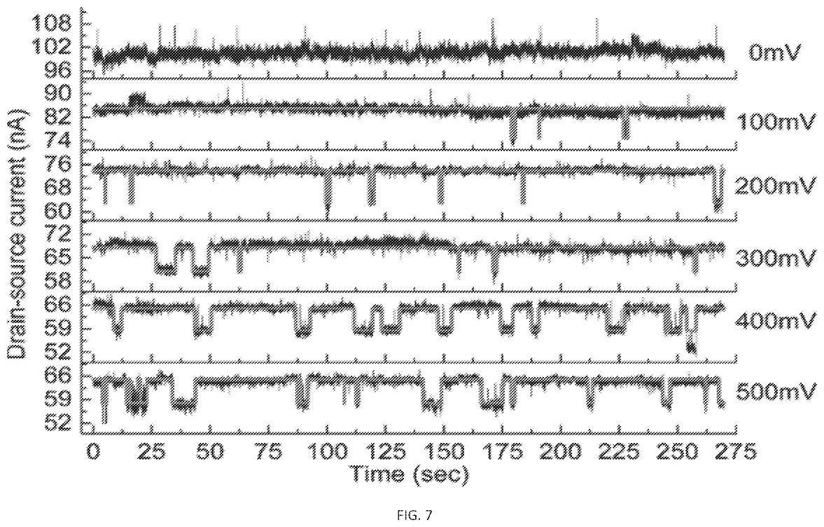

The smFET platform (FIG. 6a) uses a carbon-nanotube-based field-effect transistor sensor, where a single molecular probe is attached to a point defect in a single carbon nanotube. This setup produces an ultrahigh signal-to-noise ratio on an isolated sensor, compared to traditional nanotube and nanowires that are sensitive to field effect over their entire lengths (resulting in suboptimal limits of detection, with the lowest at 14 pM for DNA). In smFET, whether the donor DNA is bound or released is encoded at two discrete current levels (e.g., digital) with temporal recordings revealing dwell times at the levels, rather than by measuring amplitudes (e.g., analog). The “digital” design renders the platform immune to factors that corrupt signal amplitudes, such as non-specific interactions. Ultrasensitive detection of DNA hybridization and of conformational changes, as well as protein activity, have been accomplished. The smFET sensors can occupy individual sites on an integrated circuit footprint.

Several important innovations realized these devices, including the ability to make point defects in a manufacturable process, the ability to analyze the temporal data from these devices, and the ability to modulate thermodynamics with electrostatics.

Point-defects on the smFET device through controlled diazonium point functionalization. To use these nanotube devices as smFETs, a single covalent attachment of a desired biomolecule to the single-walled carbon nanotube (SWCNT) is controllably introduced. Point-functionalization approach that is electrically controllable is used, such that many devices in an array can be functionalized in parallel. Building on recent studies suggesting that the number of sp³ defects on individually contacted CVD-grown SWCNT devices induced by diazonzium salt chemistry can be tuned by changing the gate voltage. SWCNT devices are exposed to 4-formylbenzene diazonium hexafluorophosphate (FBDP), which generates an aldehyde group after the diazonium reaction (FIG. 6A). Electron transfer from the carbon nanotube (CNT) to the diazonium molecule is controlled through the applied solution potential (V_{Ig}), which modulates the Fermi energy of the CNT and the rate at which diazonium attachment occurs. This permits one to monitor the creation of a single defect in real-time and immediately decrease the solution potential relative to the nanotube to halt the reaction once a defect has been generated. A countable number of discrete current drops are easily observed (FIG. 6B), attributed to individual defect generation.

This point functionalization gives the devices two key attributes required of single-molecule sensors not present on undefected nanotubes—localized charge sensitivity and high gain-bringing almost perfect reproducibility to functional devices and producing millions of electrons of signal from each small molecule temporarily captured by the probe. Before functionalization, the nanotube is sensitive to field-effect over its entire length. After functionalization, this gain is localized to the point of functionalization.

After the functionalization stage, probes are attached. For DNA probes, smFETs are exposed to the probe DNA in 100 mM sodium phosphate buffer solution with pH 8.0 with 20011M sodium cyanoborohydride (NaBH₃CN) dissolved in 1 M NaOH, which is used to reduce the Schiff base formed between the amine and aldehyde, rendering a stable secondary amine. In the case of DNA hybridization, the resulting defect-dominated conductance produces time-domain signals as shown in the inset of FIG. 1C in which conductance fluctuates between two levels associated with hybridized and melted states are observed in the presence of complementary target.

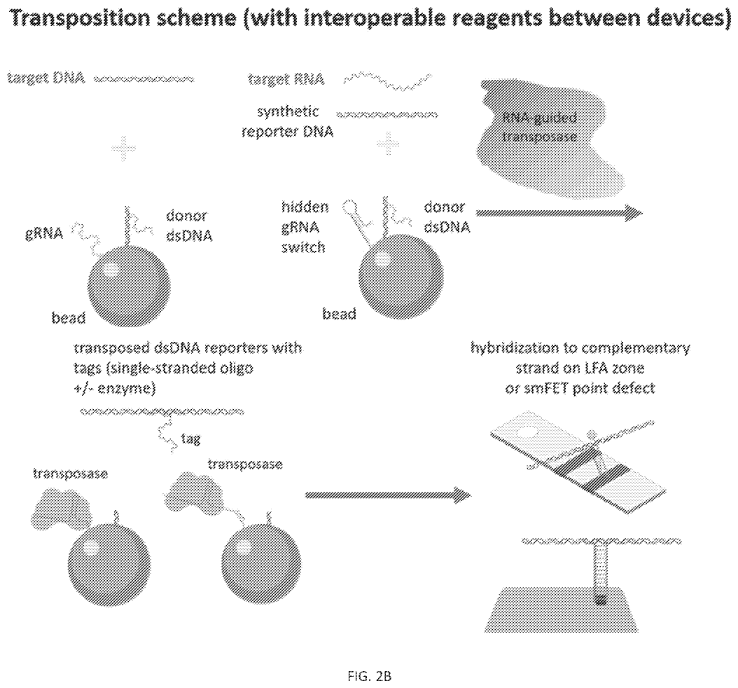

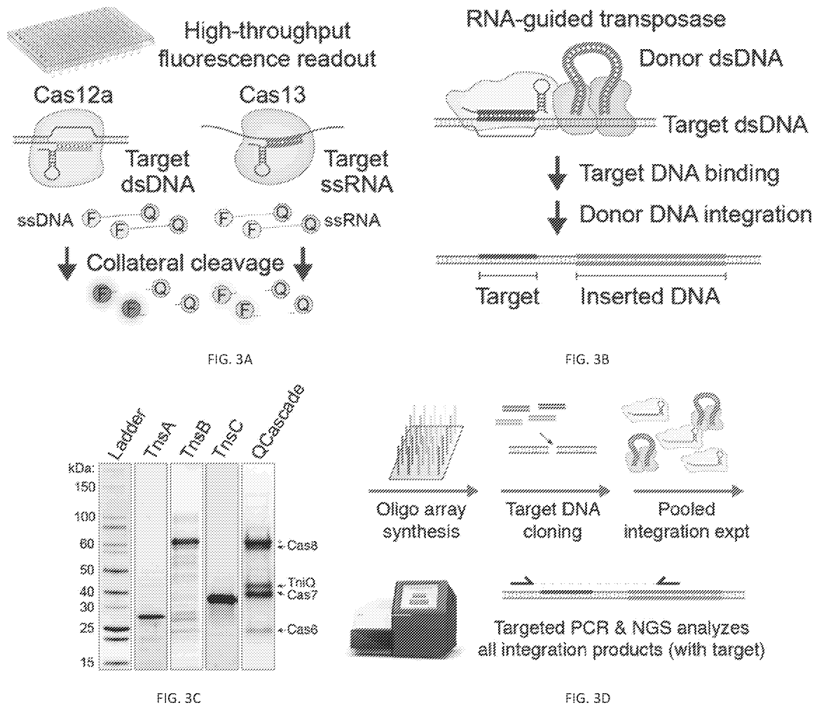

Temporal analysis of time-series data. A key attribute of the smFET device is the temporal encoding of information in the dwell times associated with each of the states. Dwell times in high (τ_{high}) and low states (τ_{low}) are extracted in the presence of flicker noise by idealizing the transitions using a hidden Markov model-based analysis approach. As shown in FIG. 3, dwell time histograms are extracted from the data and fit (usually with a double exponential) to determine time constants (and their associated reciprocal rate constants). From chemical kinetics, the solution hybridization rate (k_{hybridizing}) is proportional to DNA target concentration and the solution melting rate (k_{melting}) is independent of concentration, k_{hybridizing} extrapolated from measured data for the 10-mer DNA probe using a single smFET sensor predicts an average capture time of approximately 55 minutes at 100fM. One of the key benefits of supporting large smFET arrays is to provide the opportunity for many replicates of the same probe. The time to detection will scale linearly with the number of smFET devices participating in the detection. If an average of 55 minutes is needed for a single capture event with one sensor, having 11 probe replicates would reduce this detection time to 5 minutes. For the 1000-plex array, at least 11 probe replicates for each target can be achieved in one array.

Using bias to adjust melting temperatures. To immobilize specific oligonucleotide probes to individual smFETs, desired smFETs capable of binding a probe are voltage selected, relying on the fact that the reaction rate of probe binding is determined by voltage. Electrostatic modulation of hybridization and melting kinetics allows bias to act as a proxy for temperature, with electrostatic melting (e-melting) possible at a fixed temperature, alleviating the need to operate smFET devices at elevated temperatures or as a function of temperature changes. In addition, electrostatic repulsion between the surface of the SWCNT and the probe-DNA-target-DNA hybrid destabilizes metastable misaligned intermediates which tend to depress hybridization rates at high target concentrations. The ability to adjust the effective melting temperature using bias has been demonstrated. These data show that the melting temperature can be modulated by bias, allowing the effective temperature to be adjusted at each array site even though the entire array is operating at a fixed temperature.

The oligonucleotides are attached to smFET using diazonium chemistry, whereby the surface donates an electron to the positively-charged N, group to form a covalent amine-aldehyde bond. The probes are dried with stabilization reagents.

FET Multiplexor: Fluidic operation. A hydraulic microfluidic valve system (FIG. 1), with rapid (˜50 ms) response times and coordinated switching of multiple valves is used. Valves are placed before and after the single fluidic chamber that encloses the entire array of smFETs. Optionally, the design may also include sequentially placed valves that can act as local pumps to stir the fluid, to induce rapid kinetics (the gain in heterogeneous binding kinetics in microfluidics has been modeled). The instrument powers a pump for fluid flow. To operate the FET Multiplexor, sample handling for the user is similar to that of FET Strip, but with one fewer step and larger volume. After lysis, the 1.5 mL of fluid is transferred straight to the Multiplexor chip, where it first solubilizes dried Cas-transposase and synthetic reporter dsDNA, and continues to the main single fluidic chamber, inside which DNA and RNA targets are exposed to all 1000 recognition sequences for up to 10 minutes. Here, transposition events with the gRNA localized to each FET are directly detected (Table 7).

Whereas smFETs can detect the release of single DNA molecules from the surface, employing a 2′— or 3′-fluoro substitution generates an even larger alteration in tranconductance, due to close apposition of a highly electronegative species that presents electrostatic effects in a low-dimension channel. The integrity of the solvation shell around the fluorine atom may impart effects at greater distances (e.g., protruding into the Debye radius of the sensing voxel) to generate additional transconductance changes. As there is no splitting or compartmentalization of samples, the required fM sensitivity (˜10 copies/150 μL) and statistics of target recognition will not change significantly upon multiplexing. Detection of transposition events are direct and ultrasensitive.

TABLE 7
Identification of targets in FET Multiplexor.

| | | Bead type that | | | |
|---|---|---|---|---|---|
| | Target | recognizes target | The bead | Reporter | |
| Target | sequence | sequence would | type also | sequence in | |
| number | present | contain . . . | contains . . . | solution | FET |
| 1 | adenovirus | “gRNA 1” against | reporter | cleavage of | electronic signal |
| (Cas 12a) | dsDNA | target 1 | DNA with | ssDNA linker to | in zone 1 (which |
| | | | HRP and | release dsDNA | captures “oligo |
| | | | “oligo tag | reporter with HRP | tag 1”) |
| | | | 1” | and ssRNA “oligo | |
| | | | | tag 1” | |
| 2 | influenza | “gRNA 2” against | reporter | cleavage of | electronic signal |
| (Cas 13) | ssRNA | target 2 | DNA with | ssRNA linker to | in zone 2 (which |
| | | | HRP and | release dsDNA | captures “oligo |
| | | | “oligo tag | reporter with HRP | tag 2”) |
| | | | 2” | and ssDNA “oligo | |
| | | | | tag 2” | |
| 1 | adenovirus | “gRNA 1” against | donor | adenovirus | electronic signal |
| (transposition) | dsDNA | target 1 | DNA with | dsDNA with HRP | in zone 1 (which |
| | | | HRP and | and ssDNA “oligo | captures “oligo |
| | | | “oligo tag | tag 1” inserted | tag 1”) |
| | | | 1” | | |
| 2 | influenza | “RNA switch” with | donor | synthetic reporter | electronic signal |
| (transposition) | ssRNA | detection sequence | DNA with | dsDNA with HRP | in zone 2 (winch |
| | | against target 2. | HRP and | and ssDNA “oligo | captures “oligo |
| | | “gRNA 2” on the | “oligo tag | tag 2” inserted | tag 2”) |
| | | switch is exposed. | 2” | | |

2 influenza “RNA switch” with donor synthetic reporter electronic signal (transposition) ssRNA detection sequence DNA with dsDNA with HRP in zone 2 (which against target 2. HRP and and ssDNA “oligo captures “oligo “gRNA 2” on the “oligo tag tag 2” inserted tag 2”) switch is exposed. 2”

Software. The Multiplexor instrument hosts software for algorithmic analysis with clear digital display of results (e.g., identity and levels of the pathogens detected, and severity assessment), as well as capability to upload results to a biosurveillance network. A battery-powered instrument that ran all fluidic, electronic, and remote communication capabilities (cell phone and satellite) on microfluidic chips has been demonstrated as tested in sub-Saharan Africa. In addition, geospatial information has been integrated, and user-friendly interfaces have been built and field-tested by untrained users with detailed usability studies.

To interpret the multiplexed data, positive and negative cut-offs are defined for each target based on limit-of-detection (LOD) measurements. Commonly in diagnostic assays cut-offs are between 3 to 5 times the LOD. Positive signals are scored first for the conserved pathogen-specific targets to identify the disease agent. Next, additional probes are analyzed for sub—, sero—, and genotyping. In the case of bacteria, the presence or absence of antimicrobial genes (if the targets arc present in the panel) is scored to anticipate a potential resistance profile. In some instances, the true phenotypic expression of resistance cannot be deduced from the mere presence of a resistance gene. Thus, the resulting profile may be a first approximation, but can still guide therapy toward treatments for which no resistance gene is recorded. The disease agent(s) detected to the biomarker probe panel are linked to deduce disease severity and prognosis for the individual patient. This analysis allows the user to clear triage with respect to treatment options (such as antibiotics), and probable treatment intensity and duration.

The results are updated to a custom cloud server, for real-time remote monitoring of disease outbreak data. Moreover, the output data is in formats compatible with the Global Emerging Infections Surveillance from the DoD and/or the National Notifiable Diseases Surveillance System from the CDC, such that they can be easily integrated into the networks in the future.

It is understood that the foregoing detailed description and accompanying examples are merely illustrative and are not to be taken as limitations upon the scope of the disclosure, which is defined solely by the appended claims and their equivalents. All publications and patents mentioned in the above specification are herein incorporated by reference as if expressly set forth herein. Various changes and modifications to the disclosed embodiments will be apparent to those skilled in the art and may be made without departing from the spirit and scope thereof.

What is claimed is:

1. A system comprising:
(a) a reporter nucleic acid comprising a tag;
(b) at least one or both of: a guide RNA that comprises a nucleic acid sequence complementary to a gRNA target nucleic acid sequence and an RNA switch; and
(c) a CRISPR RNA-guided Cas-transposase system, or a Cas protein selected from Cas 12a, Cas 13, and combinations thereof.

2. The system of claim 1, wherein the reporter nucleic acid comprises dsDNA.

3. The system of claim 1 or 2, wherein the tag is an enzyme, an affinity tag, a single-stranded oligonucleotide, a halogen, or any combination thereof conjugated to the reporter nucleic acid.

4. The system of any of claims 1-3, wherein the CRISPR RNA-guided Cas-transposase system comprises at least one Cas protein, at least one transposase protein, or combinations thereof.

5. The system of any of claims 1-4, wherein the CRISPR RNA-guided Cas-transposase system is derived from Type I CRISPR-Cas system or a Type V CRISPR-Cas system.

6. The system of any of claims 1-5, wherein the CRISPR RNA-guided Cas-transposase system comprises Cas12k.

7. The system of any of claims 1-5, wherein the CRISPR RNA-guided Cas-transposase system comprises Cas5, Cas6, Cas7, Cas8, or any combination thereof.

8. The system of any of claims 1-7, wherein the CRISPR RNA-guided Cas-transposase system comprises TnsA, TnsB, TnsC, or any combination thereof.

9. The system of any of claims 1-8, further comprising a target nucleic acid.

10. The system of claim 9, wherein the target nucleic acid is double-stranded DNA or single-stranded RNA.

11. The system of claim 10, wherein the single-stranded RNA target nucleic acid is complementary to at least a portion of the RNA switch.

12. The system of claim 10, wherein the double-stranded DNA target nucleic acid is configured to receive the reporter nucleic acid as a result of a transposition.

13. The system of claim 12, wherein the double-stranded DNA target nucleic acid comprises the gRNA target nucleic acid sequence.

14. The system of any of claims 9-13, wherein the target nucleic acid is in a biological sample.

15. The system of any of claims 9-14, wherein the target nucleic acid is a nucleic acid from or derived from an infectious agent.

16. The system of any of claims 1-15 further comprising a recipient nucleic acid.

17. The system of claim 16, wherein the recipient nucleic acid is double-stranded DNA.

18. The system of claim 16 or claim 17, wherein the recipient nucleic acid is configured to receive the reporter nucleic acid as a result of a transposition.

19. The system of any of claims 16-18, wherein the recipient nucleic acid comprises the gRNA target nucleic acid sequence.

20. The system of any of claims 1-19, wherein the reporter nucleic acid, the guide RNA, the RNA switch, or any combination thereof are conjugated to a surface.

21. The system of claim 20, wherein the reporter nucleic acid, the guide RNA, the RNA switch, or any combination thereof are conjugated to a microparticle.

22. The system of claim 21, wherein the reporter nucleic acid and the guide RNA or the RNA switch are conjugated to the same microparticle.

23. The system of claim 21 or claim 22, wherein the reporter nucleic acid is conjugated to the microparticle with a linker comprising a single-stranded polynucleotide.

24. The system of any of claims 3-23, further comprising an indicator nucleic acid, wherein at least a portion of the indicator nucleic acid is complementary to at least a portion of the reporter nucleic acid or the single-stranded oligonucleotide tag.

25. The system of any of claims 1-24, further comprising a linear flow assay strip.

26. The system of claim 25, wherein the linear flow assay strip comprises a membrane and a sample filter pad on top of a portion of the membrane at first end, wherein the sample filter pad is comprised of pores smaller than those of the microparticle.

27. The system of 26, wherein the indicator nucleic acid is immobilized in the membrane of the linear flow assay strip.

28. The system of any of claims 1-23, further comprising a multiplexor chip.

29. The system of claim 28, wherein the reporter nucleic acid, the guide RNA, the RNA switch, or any combination thereof are conjugated to the multiplexor chip.

30. The system of claim 28 or 29, wherein the reporter nucleic acid is conjugated to the multiplexor chip.

31. The system of any of claims 28-30, further comprising a field-effect transistor biosensor.

32. The system of claim 31, wherein the field-effect transistor biosensor is a single-molecule field-effect transistor sensor.

33. A method of detecting a nucleic acid, comprising:
obtaining a target nucleic acid;
incubating the sample with:
a reporter nucleic acid comprising a tag,
at least one or both of: a guide RNA that comprises a nucleic acid sequence complementary to a target DNA sequence and an RNA switch, and
a CRISPR RNA-guided Cas-transposase system, or a Cas protein selected from Cas 12a, Cas 13, and combinations thereof to form a sample mixture; and
measuring the presence of the reporter nucleic acid on a linear flow assay strip or a multiplexor chip.

34. The method of claim 33, wherein the target nucleic acid is double-stranded DNA or single-stranded RNA.

35. The method of claim 34, wherein the single-stranded RNA target nucleic acid is complementary to at least a portion of the RNA switch.

36. The method of claim 34, wherein the double-stranded DNA target nucleic acid is configured to receive the reporter nucleic acid as a result of a transposition.

37. The method of claim 36, wherein the double-stranded DNA target nucleic acid comprises the gRNA target nucleic acid sequence.

38. The method of any of claims 33-37, wherein the target nucleic acid is in a biological sample.

39. The method of any of claims 33-38, wherein the target nucleic acid is a nucleic acid from or derived from an infectious agent.

40. The method of any of claims 33-39, wherein the reporter nucleic acid comprises dsDNA.

41. The method of any of claims 33-40, wherein the tag is an enzyme, an affinity tag, a single-stranded oligonucleotide, a halogen, or any combination thereof conjugated to the reporter nucleic acid.

42. The method of any of claims 33-41, wherein the CRISPR RNA-guided Cas-transposase system comprises at least one Cas protein, at least one transposase protein, or combinations thereof.

43. The method of any of claims 33-42, wherein the CRISPR RNA-guided Cas-transposase system is derived from Type I CRISPR-Cas system or a Type V CRISPR-Cas system.

44. The method of any of claims 33-43, wherein the CRISPR RNA-guided Cas-transposase system comprises Cas12k.

45. The method of any of claims 33-43, wherein the CRISPR RNA-guided Cas-transposase system comprises Cas5, Cash, Cas7, Cas8, or any combination thereof.

46. The method of any of claims 33-45, wherein the CRISPR RNA-guided Cas-transposase system comprises TnsA, TnsB, TnsC, or any combination thereof.

47. The method of claim 33-46, further comprising incubating the sample with a recipient nucleic acid.

48. The method of claim 47, wherein the recipient nucleic acid is double-stranded DNA.

49. The method of claim 47 or claim 48, wherein the recipient nucleic acid is configured to receive the reporter nucleic acid as a result of a transposition.

50. The method of any of claims 47-49, wherein the recipient nucleic acid comprises the gRNA target nucleic acid sequence.

51. The method of any of claims 33-50, further comprising incubating the sample mixture with an indicator nucleic acid.

52. The method of claim 51, wherein at least a portion of the indicator nucleic acid is complementary to at least a portion of the reporter nucleic acid or the single-stranded oligonucleotide tag.

53. The method of any of claims 33-52, wherein the reporter nucleic acid, the guide RNA, the RNA switch, or any combination thereof are conjugated to a surface.

54. The method of claim 54, wherein the reporter nucleic acid, the guide RNA, the RNA switch, or any combination thereof are conjugated to a microparticle.

55. The method of claim 54, wherein the reporter nucleic acid and the guide RNA or the RNA switch are conjugated to the same microparticle.

56. The method of claim 54 or claim 55, wherein the reporter nucleic acid is conjugated to the microparticle with a linker comprising a single-stranded polynucleotide.

57. The method of any of claims 33-56, wherein the linear flow assay strip comprises a membrane and a sample filter pad on top of a portion of the membrane at first end, wherein the sample filter pad is comprised of pores smaller than those of the microparticle.

58. The method of 57, wherein the indicator nucleic acid is immobilized in the membrane of the linear flow assay strip.

59. The method of any of claims 33-58, wherein the reporter nucleic acid, the guide RNA, the RNA switch, or any combination thereof are conjugated to the multiplexor chip.

60. The method of any of claims 28-30, wherein the multiplexor chip comprises a field-effect transistor biosensor.

61. The method of claim 60, wherein the field-effect transistor biosensor is a single-molecule field-effect transistor sensor.


SEQUENCE LISTING (3 sequences)

SEQ ID NO: 1 - RNA, 19 nt, Artificial Sequence
  Synthetic
aauuucuacucuuguagau

SEQ ID NO: 2 - RNA, 28 nt, Artificial Sequence
  Synthetic
aagauagcccaagaaagagggcaauaac

SEQ ID NO: 3 - RNA, 20 nt, Artificial Sequence
  Synthetic
gugaacugccgaguagguag